(19) United States
(12) Patent Application Publication
(10) Pub. No.: US 2009/0275124 A1
(43) Pub. Date: Nov. 5, 2009

(54) Methods and Compositions Comprising Anti-Idiotypic Antibodies to Anti-MMP-14 Antibodies

(75) Inventors: Arumugam Muruganandam, Bangalore (IN); Christopher Tenhoor, Hopkinton, MA (US); Laetitia Devy, Somerville, MA (US)

Correspondence Address: LANDO & ANASTASI, LLP, ONE MAIN STREET, SUITE 1100, CAMBRIDGE, MA, 02142, US

(73) Assignee: Dyax Corp., Cambridge, MA (US)

(21) Appl. No.: 12/429,338

(22) Filed: Apr. 24, 2009

Related U.S. Application Data
(60) Provisional application No. 61/047,787, filed on Apr. 25, 2008.

(51) Int. Cl. C12N 5/06; A61K 49/16; A61K 39/395; C12N 15/63; G01N 33/536; C07K 16/42; C07H 21/00

(52) U.S. Cl. 435/325; 424/9.1; 424/131.1; 435/320.1; 436/536; 530/387.2; 536/23.53

(57) ABSTRACT

Provided are anti-idiotypic antibodies specific for a CDR of an anti-MMP-14 antibody for use as reagents in novel assays for anti-MMP-14 antibodies, pharmaceutical compositions and vaccines.

### CROSS REFERENCE TO RELATED APPLICATIONS

This application claims priority to U.S. Application Ser. No. 61/047,787, filed on Apr. 25, 2008. The disclosures of the prior application is considered part of (and is incorporated by reference in) the disclosure of this application.

### BACKGROUND

The fully human monoclonal antibody, DX-2400, is a novel protease inhibitor that specifically inhibits matrix metalloproteinase 14 (MMP-14) on tumor cells and tumor blood vessels. DX-2400 offers a potential treatment for a broad range of solid tumors. It has been shown to significantly inhibit tumor progression and metastasis in multiple preclinical models in a dose-responsive manner when used as a monotherapy.

Interference of endogenous MMP inhibitors such as TIMP in serum prevents the use of active MMP-14 as a capture reagent in developing assays for anti-MMP-14 antibody (such as DX-2400) levels in pre-clinical and clinical sera. Enzymes in general are not sufficiently stable, especially in different serum matrices, to allow development of robust, sensitive and specific assays. Current electrochemiluminescence (ECL) methods using polyclonal rabbit anti-DX-2400 antibodies for such pharmacokinetic assays are limited to use in measuring drug levels in rodent sera and are not suitable for cynomolgus monkey and human sera.

### SUMMARY

Anti-idiotypic antibodies are ideal for developing sensitive and specific assays for drug level measurement. Provided are anti-idiotypic antibodies against anti-MMP-14 antibodies such as DX-2400 and pharmaceutical and diagnostic compositions thereof.

In certain embodiments, the anti-idiotypic antibodies are used as assay reagents in various methods. For example, the antibodies may be used as reagents for developing novel assays to determine the pharmacokinetic profile of anti-MMP-14 antibodies such as DX-2400, and to identify and characterize potential immune response directed against anti-MMP-14 antibodies such as DX-2400. They may also be used, for example, as drug-specific reagents to assess tissue biopsies in immunohistochemical methods, Western blots, and the like. Further, the antibodies may be used as affinity reagents to capture and purify anti-MMP-14 antibodies such as DX-2400 from cell culture supernatants.

In another aspect, provided are compositions of the anti-idiotypic antibodies, e.g., pharmaceutical compositions. For example, such compositions may be used as an antidote to selectively deplete DX-2400 and other anti-MMP-14 antibodies in a subject if and when there is an adverse reaction to the antibody treatment. In another example, because anti-idiotype antibodies have the potential for cognate antigen mimicry, the compositions could be used as a vaccine as well be used as a catalytic antibody.

In yet another aspect, the anti-idiotypic antibodies may be used as affinity reagents to capture and purify DX-2400 from cell culture supernatants.

Kits for the practice of these methods are also described herein.

In some aspects, the disclosure provides an isolated protein comprising a heavy chain (HC) immunoglobulin variable domain sequence and a light chain (LC) immunoglobulin variable domain sequence, wherein the HC and LC immunoglobulin variable domain sequences form an antigen binding site that binds to an anti-MMP-14 antibody; and the protein has one or more of the following characteristics:
(a) a human CDR or human framework region;
(b) the HC immunoglobulin variable domain sequence comprises one or more CDRs that are at least 85% identical to a CDR of a HC variable domain of 539C-M0016-E11; 539C-M0021-E01; 539C-R0010-A01; 539C-R10-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04;
(c) the LC immunoglobulin variable domain sequence comprises one or more CDRs that are at least 85% identical to a CDR of a LC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04;
(d) the LC immunoglobulin variable domain sequence is at least 85% identical to a LC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04;
(e) the HC immunoglobulin variable domain sequence is at least 85% identical to a HC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04; and
(f) the protein binds an epitope that overlaps with an epitope bound by 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04.

In some embodiments, the anti-MMP-14 antibody is DX-2400.

In some aspects, the disclosure provides an isolated nucleic acid that includes a sequence that encodes a polypeptide that comprises a sequence at least 80% identical to the sequence of a variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04. In some embodiments, the disclosure provides a vector comprising the nucleic acid sequence. In some embodiments, the disclosure provides a host cell comprising the nucleic acid.

In some aspects, the disclosure provides an isolated nucleic acid comprising a sequence that encodes a polypeptide comprising the HC and/or the LC immunoglobulin variable domain of the protein of described herein. In some embodiments, the disclosure provides a vector comprising the nucleic acid sequence. In some embodiments, the disclosure provides a host cell comprising the nucleic acid.

In some aspects, the disclosure provides a method of detecting an anti-MMP-14 antibody in a biological sample. The method includes contacting the sample with a protein described herein (e.g., anti-idiotype antibody); and detecting an interaction between the protein and the anti-MMP-14 antibody if present.

In some embodiments, the anti-MMP-14 antibody is DX-2400.

In some aspects, the disclosure provides a method of detecting an anti-MMP-14 antibody in a subject. The method includes: administering the protein of claim 1, that further comprises a detectable label, to a subject; and detecting the label in the subject.

In some embodiments, the anti-MMP-14 antibody is DX-2400.

In some aspects, the disclosure provides a method of treating or preventing therapeutic antibody poisoning, the method comprising: administering a protein described herein (e.g., anti-idiotype antibody) to a subject having poisoning or at risk of developing poisoning (e.g., a subject to whom a therapeutic antibody (e.g., an anti-MMP14 antibody, e.g., DX-2400) has been administered).

In some embodiments, the therapeutic antibody is DX-2400.

In some aspects, the disclosure provides a method of purifying or removing an anti-MMP-14 antibody from a solution (e.g., a cell extract or biological sample). The method includes: contacting the solution with a protein described herein (e.g., anti-idiotype antibody); and eluting the anti-MMP-14 antibody that binds to the protein.

In some embodiments, the anti-MMP-14 antibody is DX-2400.

These embodiments of the present invention, other embodiments, and their features and characteristics will be apparent from the description, drawings, and claims that follow.

### BRIEF DESCRIPTION OF THE DRAWINGS

FIG. 1 depicts amino acid sequences of Fab heavy chain (HC) and light chain (LC) variable regions of some exemplary anti-MMP14 antibodies against which the anti-idiotypic antibodies described herein may be raised. The standard numbering of the HC V domain is shown. The length of HC CDR3 varies considerably. By convention, the second cysteine is numbered 92 and the W of the conserved WG motif of FR4 is number 103. If there are more than 9 residues between C92 and W103, then residues after 102 are numbered 102a, 102b, etc.

FIGS. 2A and 2B. FIG. 2A is a diagram depicting the ECL assay format used in Example 3. FIG. 2B is a line graph showing a representative standard curve in 1.25% mouse serum.

FIG. 3 is a bar graph showing the detected concentration of DX-2400 as calculated by interpolation from the calibration curve made in FIG. 2B.

FIG. 4 is a flow chart of the Bioanalytical Assay Development.

FIG. 5 is diagrams showing the phage-ELISA screening.

FIG. 6 is a series of four bar graphs showing the specificity assessment in the presence of 5% serum matrices (Phage ELISA).

FIG. 7 is series of tables showing the specificity ranking of phage candidates.

FIG. 8 is a diagram depicting the ELISA format used for anti-idiotype Fab screening.

FIGS. 9A and 9B. FIG. 9A is a diagram showing the assay format used. FIG. 9B is a line graph showing representative standard curves of DX2400.

### DETAILED DESCRIPTION

Human anti-idiotypic antibodies against anti-MMP-14 antibodies (e.g., DX-2400) of the present disclosure are useful, for example, for developing pre-clinical and clinical bioanalytical pharamacokinetic (PK), immune response (IR) and neutralizing antibody (NAb) assays.

For example:
1. Anti-idiotypic antibodies(anti-Ids) against anti-MMP-14 antibodies (e.g., DX-2400) can be used as assay reagents for developing novel and innovative bioanalytical assays to determine the pharmacokinetic (PK) profile of, and to identify and characterize, potential immune response (IR) directed against the protein (e.g., MMP-14).
2. Since anti-MMP-14 antibodies in the human body can have a long half-life (e.g., DX-2400 has a have life of approximately ˜21 days), anti-idiotypic antibodies can be used as an antidote to selectively deplete anti-MMP-14 antibodies (e.g., DX-2400) in the human body if and when there is an adverse reaction to the antibody.
3. Anti-Ids have the potential for cognate antigen mimicry as such the anti-Ids paratope could be used as a vaccine.
4. Anti-Ids can be used as an affinity reagent to capture and purify anti-MMP-14 antibodies (e.g., DX-2400) from solution, e.g., from cell culture supernatants.
5. Anti-Ids can be used as drug specific reagent to assess tumor biopsies by immunohistochemistry and western blots.

For convenience, before further description of the present invention, certain terms employed in the specification, examples and appended claims are defined here.

The singular forms “a”, “an”, and “the” include plural references unless the context clearly dictates otherwise.

An “anti-idiotypic antibody” is an antibody directed against the antigen specific part of the sequence of an antibody, i.e., the CDR (as defined below), and thus is an antibody that recognizes the antigen-specific binding sites of other antibodies.

The term “antibody” refers to any protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J. Immunol. 1996; 26(3):629-39.)) as well as complete antibodies. An antibody can have the structural features of IgA, IgG, IgE, IgD, IgM (as well as subtypes thereof). Antibodies may be from any source, but primate (human and non-human primate) and primatized are preferred.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system. The light chains of the immunoglobulin may be of types kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and LC CDR3. Each of the light chain CDRs can be human. HC CDR3 can be human. One or more of the framework regions can be human, e.g., FR1, FR2, FR3, and FR4 of the HC or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, CL1), or the entire antibody can be human or effectively human.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

An “anti-MMP14 antibody” refers to an antibody raised against an MMP-14 antigen. MMP-14 is encoded by a gene designated as MMP14, matrix metalloproteinase-14 precursor. Synonyms for MMP-14 include matrix metalloproteinase 14 (membrane-inserted), membrane-type-1 matrix metalloproteinase, membrane-type matrix metalloproteinase 1, MMP14, MMP-X1, MT1MMP, MT1-MMP, MTMMP1, MT-MMP 1. MT-MMPs have similar structures, including a signal peptide, a prodomain, a catalytic domain, a hinge region, and a hemopexin domain (Wang, et al., 2004, J Biol Chem, 279:51148-55). According to SwissProt entry P50281, the signal sequence of MMP-14 precursor includes amino acid residues 1-20. The pro-peptide includes residues 21-111. Cys93 is annotated as a possible cysteine switch. Residues 112 through 582 make up the mature, active protein. The catalytic domain includes residues 112-317. The hemopexin domains includes residues 318-523. The transmembrane segment comprises residues 542 through 562.

An exemplary amino acid sequence of human MMP-14 is shown in Table 1:

TABLE 1
Amino-acid sequence of human MMP-14

MSPAPRPPRCLLLPLLTLGTALASLGSAQSSSFSPEAWLQQYGYLPPGDL
RTHTQRSPQSLSAAIAAMQKFYGLQVTGKADADTMKAMRRPRCGVPDKFG
AEIKANVRRKRYAIQGLKWQHNEITFCIQNYTPKVGEYATYEAIRKAFRV
WESATPLRFREVPYAYIREGHEKQADIMIFFAEGFHGDSTPFDGEGGFLA
HAYFPGPNIGGDTHFDSAEPWTVRNEDLNGNDIFLVAVHELGHALGLEHS
SDPSAIMAPFYQWMDTENFVLPDDDRRGIQQLYGGESGFPTKMPPQPRTT
SRPSVPDKPKNPTYGPNICDGNFDTVAMLRGEMFVFKERWFWRVRNNQVM
DGYPMPIGQFWRGLPASINTAYERKDGKFVFFKGDKHWVFDEASLEPGYP
KHIKELGRGLPTDKIDAALFWMPNGKTYFFRGNKYYRFNEELRAVDSEYP
KNIKVWEGIPESPRGSFMGSDEVFTYFYKGNKYWKFNNQKLKVEPGYPKS
ALRDWMGCPSGGRPDEGTEEETEVIIIEVDEEGGGAVSAAAVVLPVLLLL
LVLAVGLAVFFFRRHGTPRRLLYCQRSLLDKV (SEQ ID NO:2;
Genbank Accession No. CAA88372.1).

An exemplary amino acid sequence of mouse MMP-14 is shown in Table 2.

TABLE 2
Amino-acid sequence of mouse MMP-14

MSPAPRPSRSLLLPLLTLGTALASLGWAQGSNFSPEAWLQQYGYLPPGDL
RTHTQRSPQSLSAAIAAMQKFYGLQVTGKADLATMMAMRRPRCGVPDKFG
TEIKANVRRKRYAIQGLKWQHNEITFCIQNYTPKVGEYATFEAIRKAFRV
WESATPLRFREVPYAYIREGHEKQADIMILFAEGFHGDSTPFDGEGGFLA
HAYFPGPNIGGDTHFDSAEPWTVQNEDLNGNDIFLVAVHELGHALGLEHS
NDPSAIMSPFYQWMDTENFVLPDDDRRGIQQLYGSKSGSPTKMPPQPRTT
SRPSVPDKPKNPAYGPNICDGNFDTVAMLRGEMFVFKERWFWRVRNNQVM
DGYPMPIGQEWRGLPASINTAYERKDGKFVFFKGDKHWVFDEASLEPGYP
KHIKELGRGLPTDKIDAALFWMPNGKTYFFRGNKYYRFNEEFRAVDSEYP
KNIKVWEGIPESPRGSFMGSDEVFTYFYKGNKYWKFNNQKLKVEPGYPKS
ALRDWMGCPSGRRPDEGTEEETEVIIIEVDEEGSGAVSAAAVVLPVLLLL
LVLAVGLAVFFFRRHGTPKRLLYCQRSLLDKV SEQ ID NO:4;
GenBank Accession No. NP_032634.2.

An exemplary MMP-14 protein against which anti-MMP-14 antibodies may be developed can include the human or mouse MMP-14 amino acid sequence, a sequence that is 80%, 85%, 90%, 95%, 96%, 97%, 98%, or 99% identical to one of these sequences, or a fragment thereof, e.g., a fragment without the signal sequence or prodomain.

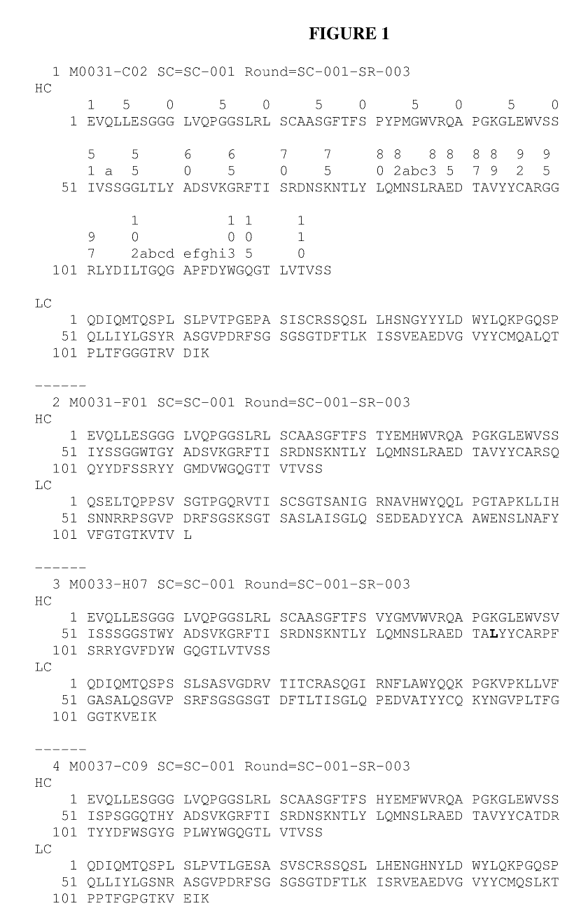

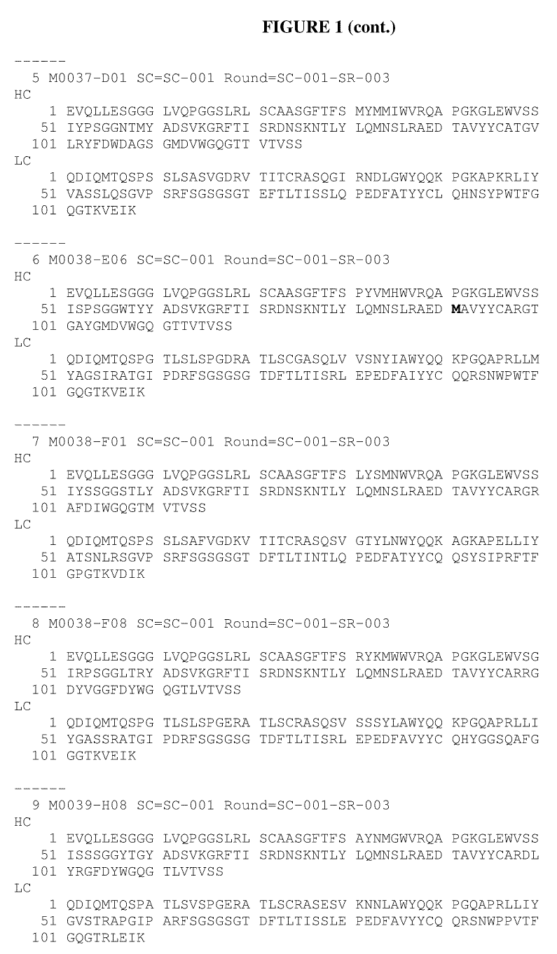

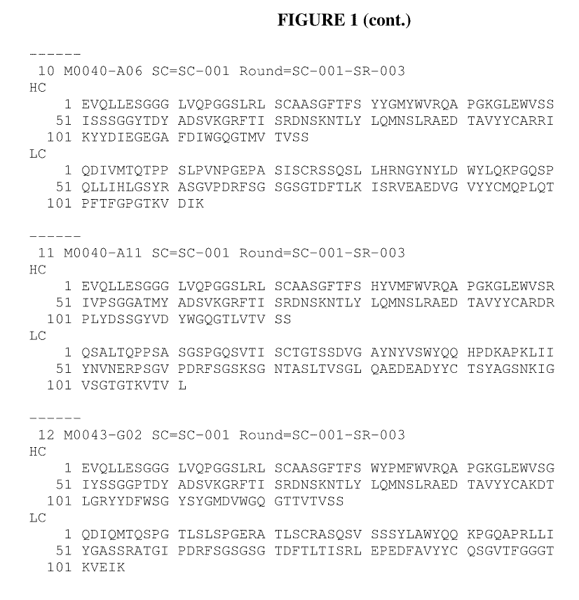

Exemplary anti-MMP-14 antibodies include M0031-C02, M0031-F01, M0033-H07, M0037-C09, M0037-D01, M0038-E06, M0038-F01, M0038-F08, M0039-H08, M0040-A06, M0040-A11, and M0043-G02. The amino acid sequences of exemplary Fab heavy chain (HC) and light chain (LC) variable regions of these binding proteins are shown in FIG. 1, and further description of them and their discovery and production is provided in pending applications U.S. Ser. No. 11/648,423 (US 2007-0217997) and also WO 2007/079218.

The term “binding” refers to an association, which may be a stable association, between two molecules, e.g., between a polypeptide of the invention and a binding partner, due to, for example, electrostatic, hydrophobic, ionic and/or hydrogen-bond interactions under physiological conditions.

“Biological activity” or “bioactivity” or “activity” or “biological function”, which are used interchangeably, refer to an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity may be modulated by directly affecting the subject polypeptide. Alternatively, a bioactivity may be altered by modulating the level of the polypeptide, such as by modulating expression of the corresponding gene.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

“Gene” or “recombinant gene” refers to a nucleic acid molecule comprising an open reading frame and including at least one exon and (optionally) an intron sequence. “Intron” refers to a DNA sequence present in a given gene which is spliced out during mRNA maturation.

The terms “label” or “labeled” refer to incorporation or attachment, optionally covalently or non-covalently, of a detectable marker into a molecule, such as a polypeptide and especially an antibody. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance. Particular examples of labels which may be used under the invention include fluorescein, rhodamine, dansyl, umbelliferone, Texas red, luminol, NADPH, alpha-beta-galactosidase and horseradish peroxidase.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

The term “modulator” refers to a polypeptide, nucleic acid, macromolecule, complex, molecule, small molecule, compound, species or the like (naturally-occurring or non-naturally-occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

A “patient”, “subject” or “host” to be treated by the subject method may mean either a human or non-human animal.

“Protein”, “polypeptide” and “peptide” are used interchangeably herein when referring to a gene product, e.g., as may be encoded by a coding sequence. By “gene product” it is meant a molecule that is produced as a result of transcription of a gene. Gene products include RNA molecules transcribed from a gene, as well as proteins translated from such transcripts.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

The term “therapeutically effective amount” refers to that amount of a modulator, drug or other molecule which is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “treating” as used herein is intended to encompass curing as well as ameliorating at least one symptom of any condition or disease.

Anti-Idiotypic Antibodies Specific for Anti-MMP-14 Antibodies

This disclosure provides anti-idiotypic antibodies that are specific for anti-MMP-14 (e.g., human MMP-14) antibodies. For example, an anti-idiotypic antibody specific for anti-MMP-14 antibody may include a heavy chain (HC) immunoglobulin variable domain sequence and a light chain (LC) immunoglobulin variable domain sequence. A number of exemplary anti-idiotypic antibodies specific for an anti-MMP-14 antibody are described herein. The anti-idiotypic antibody specific for an anti-MMP-14 antibody may be an isolated protein (e.g., at least 70, 80, 90, 95, or 99% free of other proteins).

Exemplary anti-idiotypic antibodies specific for an anti-MMP-14 antibody (DX-2400) include 539C-M0016-E11 and 539C-M0021-E01. Additional exemplary anti-idiotypic antibodies specific for an anti-MMP-14 antibody (DX-2400) include 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; and 539C-R0004-G04. The antibodies may have their HC and LC variable domain sequences included in a single polypeptide (e.g., scFv), or on different polypeptides (e.g., IgG or Fab). The antibody can include one or more of the following characteristics: (a) a human CDR or human framework region; (b) the HC immunoglobulin variable domain sequence comprises one or more CDRs that are at least 85, 88, 90, 92, 94, 95, 96, 97, 98, 99, or 100% identical to a CDR of a HC variable domain described herein; (c) the LC immunoglobulin variable domain sequence comprises one or more CDRs that are at least 85, 88, 90, 92, 94, 95, 96, 97, 98, 99, or 100% identical to a CDR of a LC variable domain described herein; (d) the LC immunoglobulin variable domain sequence is at least 85, 88, 90, 92, 94, 95, 96, 97, 98, 99, or 100% identical to a LC variable domain described herein; (e) the HC immunoglobulin variable domain sequence is at least 85, 88, 90, 92, 94, 95, 96, 97, 98, 99, or 100% identical to a HC variable domain described herein; (f) the protein binds an epitope bound by a protein described herein, or an epitope that overlaps with such epitope; and (g) a primate CDR or primate framework region. The anti-idiotypic antibodies may bind to anti-MMP-14 antibodies with a binding affinity of at least 10⁵, 10⁶, 10⁷, 10⁸, 10⁹, 10¹⁰ and 10¹¹ M⁻¹. In one embodiment, the anti-idiotypic antibodies may bind to anti-MMP-14 antibodies with a K_{off} slower than 1×10⁻³, 5×10⁻⁴ s⁻¹, or 1×10⁻⁴ s⁻¹. In one embodiment, the anti-idiotypic antibodies may bind to anti-MMP-14 antibodies with a Kₒₙ faster than 1×10², 1×10³ or 5×10³ M⁻¹s⁻¹.

In a preferred embodiment, the anti-idiotypic antibody is a human antibody having the light and heavy chains of antibodies selected from the group of antibodies consisting of 539C-M0016-E11 and 539C-M0021-E01. In a preferred embodiment, the anti-idiotypic antibody is a human antibody having its heavy chain selected from the group of antibodies consisting of 539C-M0016-E11 and 539C-M0021-E01. In a preferred embodiment, the anti-idiotypic antibody is a human antibody having its light chain selected from the group of antibodies consisting of 539C-M0016-E11 and 539C-M0021-E01. In a preferred embodiment, the anti-idiotypic antibody is a human antibody having one or more (e.g., one, two or three) heavy chain CDRs selected from the CDRs of the heavy chain of antibody 539C-M0016-E11 or 539C-M0021-E01. In a preferred embodiment, the anti-idiotypic antibody is a human antibody having one or more (e.g., one, two or three) light chain CDRs selected from the CDRs of the light chain of antibody 539C-M0016-E11 or 539C-M0021-E01. In a preferred embodiment, the anti-idiotypic antibody is a human antibody having light chain and heavy chain CDRs selected from the CDRs of both the light chain and heavy chain of antibody 539C-M0016-E11 or 539C-M0021-E01.

In a preferred embodiment, the anti-idiotypic antibody is a human antibody having the light and heavy chains of antibodies selected from the group of antibodies consisting of 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; and 539C-R0004-G04. In a preferred embodiment, the anti-idiotypic antibody is a human antibody having its heavy chain selected from the group of antibodies consisting of 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; and 539C-R0004-G04. In a preferred embodiment, the anti-idiotypic antibody is a human antibody having its light chain selected from the group of antibodies consisting of 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; and 539C-R0004-G04. In a preferred embodiment, the anti-idiotypic antibody is a human antibody having one or more (e.g., one, two or three) heavy chain CDRs selected from the CDRs of the heavy chain of antibody 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04. In a preferred embodiment, the anti-idiotypic antibody is a human antibody having one or more (e.g., one, two or three) light chain CDRs selected from the CDRs of the light chain of antibody 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04. In a preferred embodiment, the anti-idiotypic antibody is a human antibody having light chain and heavy chain CDRs selected from the CDRs of both the light chain and heavy chain of antibody 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04.

Discovery of Anti-Idiotypic Antibodies Against Anti-MMP-14 Antibodies

Display Libraries

A display library can be used to identify anti-idiotypic antibodies to anti-MMP14 antibodies. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g. from three amino acids to over 300 amino acids. In a selection, the polypeptide component of each member of the library is probed with an anti-MMP-14 antibody, and if the polypeptide component binds specifically to the CDR regions of the anti-MMP-14 antibody, the display library member is identified, typically by retention on a support. In addition, a display library entity can include more than one polypeptide component, for example, the two polypeptide chains of a sFab.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

A variety of formats can be used for display libraries. Examples include the following.

Phage Display. One format utilizes viruses, particularly bacteriophages. This format is termed “phage display.” The protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem. 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; and Hoogenboom et al. (1991) Nuc Acid Res 19:4133-4137.

Phage display systems have been developed for filamentous phage (phage f1, fd, and M13) as well as other bacteriophage. The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In one embodiment, the fusion is to a domain of the gene III protein, e.g., the anchor domain or “stump,” (see, e.g., U.S. Pat. No. 5,658,727 for a description of the gene III protein anchor domain). It is also possible to physically associate the protein being displayed to the coat using a non-peptide linkage.

Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced.

Other Display Formats. Other display formats include cell based display (see, e.g., WO 03/029456), protein-nucleic acid fusions (see, e.g., U.S. Pat. No. 6,207,446), and ribosome display (See, e.g., Mattheakis et al. (1994) Proc. Natl. Acad. Sci. USA 91:9022 and Hanes et al. (2000) Nat. Biotechnol. 18:1287-92; Hanes et al. (2000) Methods Enzymol. 328:404-30; and Schaffitzel et al. (1999) J Immunol Methods. 231(1-2):119-35).

Scaffolds. Scaffolds for display can include: antibodies (e.g., Fab fragments, single chain Fv molecules (scFV), single domain antibodies, shark antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin and heat shock proteins; intracellular signaling domains (such as SH2 and SH3 domains); linear and constrained peptides; and linear peptide substrates. Display libraries can include synthetic and/or natural diversity. See, e.g., US 2004-0005709.

Display technology can also be used to obtain ligands, e.g., antibody ligands that bind particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Iterative Selection. In one preferred embodiment, display library technology is used in an iterative mode. A first display library is used to identify one or more ligands for a target. These identified ligands are then varied using a mutagenesis method to form a second display library. Higher affinity ligands are then selected from the second library, e.g., by using higher stringency or more competitive binding and washing conditions.

In some implementations, the mutagenesis is targeted to regions known or likely to be at the binding interface. If, for example, the identified ligands are antibodies, then mutagenesis can be directed to the CDR regions of the heavy or light chains as described herein. Further, mutagenesis can be directed to framework regions near or adjacent to the CDRs. In the case of antibodies, mutagenesis can also be limited to one or a few of the CDRs, e.g., to make precise step-wise improvements. Likewise, if the identified ligands are enzymes, mutagenesis can be directed to the active site and vicinity. Exemplary mutagenesis techniques include: error-prone PCR, recombination, DNA shuffling, site-directed mutagenesis and cassette mutagenesis.

In one example of iterative selection, the methods described herein are used to first identify a protein ligand from a display library that binds to the CDR regions of an anti-MMP-14 antibody with at least a minimal binding specificity for a target or a minimal activity, e.g., an equilibrium dissociation constant for binding of less than 1 nM, 10 nM, or 100 nM. The nucleic acid sequence encoding the initial identified protein ligands are used as a template nucleic acid for the introduction of variations, e.g., to identify a second protein ligand that has enhanced properties (e.g., binding affinity, kinetics, or stability) relative to the initial protein ligand.

Off-Rate Selection. Since a slow dissociation rate can be predictive of high affinity, particularly with respect to interactions between polypeptides and their targets, the methods described herein can be used to isolate ligands with a desired kinetic dissociation rate (e.g., reduced) for a binding interaction to a target.

To select for slow dissociating ligands from a display library, the library is contacted to an immobilized target. The immobilized target is then washed with a first solution that removes non-specifically or weakly bound biomolecules. Then the bound ligands are eluted with a second solution that includes a saturating amount of free target or a target specific high-affinity competing monoclonal antibody, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting or Screening for Specificity. The display library screening methods described herein can include a selection or screening process that discards display library members that bind to a non-target molecule. Examples of non-target molecules include streptavidin on magnetic beads, blocking agents such as bovine serum albumin, non-fat bovine milk, any capturing or target immobilizing monoclonal antibody, or non-transfected cells which do not express the anti-MMP-14 antibody target.

In one implementation, a so-called “negative selection” step is used to discriminate between the target and related non-target molecule and a related, but distinct non-target molecule. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target as well as for cross species reactivity to related targets or subunits of the target and also under different condition such as pH6 or pH 7.5. The non-target and target binding data are compared (e.g., using a computer and software) to identify library members that specifically bind to the target.

Other Expression Libraries

Other types of collections of proteins (e.g., expression libraries) can be used to identify proteins with a particular property (e.g., ability to bind the CDR of an anti-MMP-14 antibody), including, e.g., protein arrays of antibodies (see, e.g., De Wildt et al. (2000) Nat. Biotechnol. 18:989-994), lambda gt11 libraries, two-hybrid libraries and so forth.

Antibody Libraries

In one embodiment, the library presents a diverse pool of polypeptides, each of which includes an immunoglobulin domain, e.g., an immunoglobulin variable domain. Display libraries are particularly useful, for example, for identifying human or “humanized” antibodies that recognize human antigens. Such antibodies can be used as therapeutics to treat human disorders such as autoimmune disorders. Because the constant and framework regions of the antibody are human, these therapeutic antibodies may avoid themselves being recognized and targeted as antigens. The constant regions may also be optimized to recruit effector functions of the human immune system. The in vitro display selection process surmounts the inability of a normal human immune system to generate antibodies against self-antigens.

A typical antibody display library displays a polypeptide that includes a VH domain and a VL domain. An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay, 1988, Ann. Rev. Immunol. 6:381-405). The display library can display the antibody as a Fab fragment (e.g., using two polypeptide chains) or a single chain Fv (e.g., using a single polypeptide chain). Other formats can also be used.

As in the case of the Fab and other formats, the displayed antibody can include one or more constant regions as part of a light and/or heavy chain. In one embodiment, each chain includes one constant region, e.g., as in the case of a Fab. In other embodiments, additional constant regions are displayed.

Antibody libraries can be constructed by a number of processes (see, e.g., de Haard et al., 1999, J. Biol. Chem. 274:18218-30; Hoogenboom et al., 1998, Immunotechnology 4:1-20; and Hoogenboom et al., 2000, Immunol. Today 21:371-378. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible. In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al., 2000, J. Mol. Biol. 296:57-86 describe a method for constructing CDR encoding oligonucleotides using trinucleotide synthesis and a template with engineered restriction sites for accepting the oligonucleotides.

In another process, an animal, e.g., a rodent, is immunized with the anti-MMP-14 antibody. The animal is optionally boosted with the antigen to further stimulate the response. Then spleen cells are isolated from the animal, and nucleic acid encoding VH and/or VL domains is amplified and cloned for expression in the display library.

In yet another process, antibody libraries are constructed from nucleic acid amplified from naïve germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method.

Nucleic acid encoding immunoglobulin domains can be obtained from the immune cells of, e.g., a human, a primate, mouse, rabbit, camel, or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin, or pokeweed mitogen.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al., 2001, J. Immunol. 166:2228). In another embodiment, the cells are naïve.

The nucleic acid encoding an immunoglobulin variable domain can be isolated from a natural repertoire by the following exemplary method. First, RNA is isolated from the immune cell. Full length (i.e., capped) mRNAs are separated (e.g. by degrading uncapped RNAs with calf intestinal phosphatase). The cap is then removed with tobacco acid pyrophosphatase and reverse transcription is used to produce the cDNAs.

The reverse transcription of the first (antisense) strand can be done in any manner with any suitable primer. See, e.g., de Haard et al., 1999, J. Biol. Chem. 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In another embodiment, poly-dT primers may be used (and may be preferred for the heavy-chain genes).

A synthetic sequence can be ligated to the 3′ end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

Secondary Screening Methods

After selecting candidate library members that bind to a target, each candidate library member can be further analyzed, e.g., to further characterize its binding properties for the target. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. Exemplary assays for binding properties include the following.

ELISA. Proteins selected from an expression library can also be screened for a binding property using an ELISA assay. For example, each protein is contacted to a microtitre plate whose bottom surface has been coated with the target, e.g., a limiting amount of the target. The plate is washed with buffer to remove non-specifically bound polypeptides. Then the amount of the protein bound to the plate is determined by probing the plate with an antibody that can recognize the protein, e.g., a tag or constant portion of the protein. The antibody is linked to an enzyme such as alkaline phosphatase or horse radish peroxidase (HRP) which produces a calorimetric product when appropriate substrates are provided.

In the case of a protein from a display library, the protein can be purified from cells or assayed in a display library format, e.g., as a fusion to a filamentous bacteriophage coat. In another version of the ELISA assay, each protein selected from an expression library is used to coat a different well of a microtitre plate. The ELISA then proceeds using a constant target molecule to query each well.

Homogeneous Binding Assays. The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is ALPHASCREEN™ (Packard Bioscience, Meriden Conn.). ALPHASCREEN™ uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

Surface Plasmon Resonance (SPR). The binding interaction of a molecule isolated from an expression library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether, 1988, Surface Plasmons Springer Verlag; Sjolander and Urbaniczky, 1991, Anal. Chem. 63:2338-2345; Szabo et al., 1995, Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a biomolecule to a target. Such data can be used to compare different biomolecules. For example, selected proteins from an expression library can be compared to identify proteins that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

Cellular Assays. A library of candidate polypeptides/antibodies (e.g., previously identified by a display library or otherwise) can be screened for target binding on cells which transiently or stably express and display the target of interest on the cell surface.

Other Methods for Obtaining Anti-Idiotypic Antibodies Against Anti-MMP-14 Antibodies

In addition to the use of display libraries, other methods can be used to obtain a anti-idiotypic antibody against an MMP-14 antibody. For example, the CDR of an anti-MMP-14 antibody or a region thereof can be used as an antigen in a non-human animal, e.g., a rodent.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies (Mabs) derived from the genes with the desired specificity may be produced and selected. See, e.g., XENOMOUSE™, Green et al., 1994, Nat. Gen. 7:13-21; U.S. 2003-0070185, WO 96/34096, published Oct. 31, 1996, and PCT Application No. PCT/US96/05928, filed Apr. 29, 1996.

In another embodiment, a monoclonal antibody is obtained from the non-human animal, and then modified, e.g., humanized or deimmunized. Winter describes a CDR-grafting method that may be used to prepare the humanized antibodies (UK Patent Application GB 2188638A, filed on Mar. 26, 1987; U.S. Pat. No. 5,225,539. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen.

Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above. The recombinant DNA encoding the humanized antibody, or fragment thereof, can then be cloned into an appropriate expression vector.

An anti-MMP14 antibody CDR binding antibody may also be modified by specific deletion of human T cell epitopes or “deimmunization” by the methods disclosed in WO 98/52976 and WO 00/34317, the contents of which are specifically incorporated by reference herein. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible conservative substitutions are made, often but not exclusively, an amino acid common at this position in human germline antibody sequences may be used. Human germline sequences are disclosed in Tomlinson, I. A. et al., 1992, J. Mol. Biol. 227:776-798; Cook, G. P. et al., 1995, Immunol. Today Vol. 16 (5): 237-242; Chothia, D. et al., 1992, J. Mol. Bio. 227:799-817. The V BASE directory provides a comprehensive directory of human immunoglobulin variable region sequences (compiled by Tomlinson, I. A. et al. MRC Centre for Protein Engineering, Cambridge, UK). After the deimmunizing changes are identified, nucleic acids encoding V_{H} and V_{L} can be constructed by mutagenesis or other synthetic methods (e.g., de novo synthesis, cassette replacement, and so forth). Mutagenized variable sequence can, optionally, be fused to a human constant region, e.g., human IgG1 or K constant regions.

In some cases a potential T cell epitope will include residues which are known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs were eliminated by substitutions outside the CDRs. In some cases, an alteration within a CDR is the only option, and thus variants with and without this substitution should be tested. In other cases, the substitution required to remove a potential T cell epitope is at a residue position within the framework that might be critical for antibody binding. In these cases, variants with and without this substitution should be tested. Thus, in some cases several variant deimmunized heavy and light chain variable regions were designed and various heavy/light chain combinations tested in order to identify the optimal deimmunized antibody. The choice of the final deimmunized antibody can then be made by considering the binding affinity of the different variants in conjunction with the extent of deimmunization, i.e., the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Germlining Antibodies. An antibody used to treat an IgG-mediated autoimmune disease can be used for multiple administrations. Precautions that would lower the immunogenicity of the therapeutic antibody include reverting one or more non-germline amino acids in framework regions to corresponding germline amino acids (e.g., so long as binding properties are substantially retained) of the antibody (especially of Fabs).

It is possible to modify an antibody that binds a CDR of an anti-MMP-14 antibody, e.g., an anti-idiotypic antibody described herein, in order to make the variable regions of the antibody more similar to one or more germline sequences. For example, an antibody can include one, two, three, or more amino acid substitutions, e.g., in a framework, CDR, or constant region, to make it more similar to a reference germline sequence. One exemplary germlining method can include identifying one or more germline sequences that are similar (e.g., most similar in a particular database) to the sequence of the isolated antibody. Mutations (at the amino acid level) can then be made in the isolated antibody, either incrementally or in combination with other mutations. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a framework and/or constant region. For example, a germline framework and/or constant region residue can be from a germline sequence that is similar (e.g., most similar) to the non-variable region being modified. After mutagenesis, activity (e.g., binding or other functional activity) of the antibody can be evaluated to determine if the germline residue or residues are tolerated (i.e., do not abrogate activity). Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may including using two germline sequences that separately contribute to the amino-terminal portion and the carboxy-terminal portion. In other implementations more than one or two germline sequences are used, e.g., to form a consensus sequence.

In one embodiment, with respect to a particular reference variable domain sequence, e.g., a sequence described herein, a related variable domain sequence has at least 30, 40, 50, 60, 70, 80, 90, 95 or 100% of the CDR amino acid positions that are not identical to residues in the reference CDR sequences, residues that are identical to residues at corresponding positions in a human germline sequence (i.e., an amino acid sequence encoded by a human germline nucleic acid).

In one embodiment, with respect to a particular reference variable domain sequence, e.g., a sequence described herein, a related variable domain sequence has at least 30, 50, 60, 70, 80, 90 or 100% of the FR regions are identical to FR sequence from a human germline sequence, e.g., a germline sequence related to the reference variable domain sequence.

Accordingly, it is possible to isolate an antibody which has similar activity to a given antibody of interest, but is more similar to one or more germline sequences, particularly one or more human germline sequences. For example, an antibody can be at least 90, 91, 92, 93, 94, 95, 96, 97, 98, 99, or 99.5% identical to a germline sequence in a region outside the CDRs (e.g., framework regions). Further, an antibody can include at least 1, 2, 3, 4, or 5 germline residues in a CDR region, the germline residue being from a germline sequence of similar (e.g., most similar) to the variable region being modified. Germline sequences of primary interest are human germline sequences. The activity of the antibody (e.g., the binding activity) can be within a factor or 100, 10, 5, 2, 0.5, 0.1, and 0.001 of the original antibody.

Exemplary germline reference sequences for Vₖₐₚₚₐ include: O12/O2, O18/O8, A20, A30, L14, L1, L15, L4/18a, L5/L19, L8, L23, L9, L24, L11, L12, O11/O1, A17, A1, A18, A2, A19/A3, A23, A27, A11, L2/L16, L6, L20, L25, B3, B2, A26/A10, and A14. See, e.g., Tomlinson et al., 1995, EMBO J. 14(18):4628-3.

A germline reference sequence for the HC variable domain can be based on a sequence that has particular canonical structures, e.g., 1-3 structures in the H1 and H2 hypervariable loops. The canonical structures of hypervariable loops of an immunoglobulin variable domain can be inferred from its sequence, as described in Chothia et al., 1992, J. Mol. Biol. 227:799-817; Tomlinson et al., 1992, J. Mol. Biol. 227:776-798); and Tomlinson et al., 1995, EMBO J. 14(18):4628-38. Exemplary sequences with a 1-3 structure include: DP-1, DP-8, DP-12, DP-2, DP-25, DP-15, DP-7, DP-4, DP-31, DP-32, DP-33, DP-35, DP-40, 7-2, hv3005, hv3005f3, DP-46, DP-47, DP-58, DP-49, DP-50, DP-51, DP-53, and DP-54.

Production of Anti-Idiotypic Antibodies Against Anti-MMP-14 Antibodies

Standard recombinant nucleic acid methods can be used to express the anti-idiotypic antibodies described herein. Generally, a nucleic acid sequence encoding the protein ligand is cloned into a nucleic acid expression vector. Of course, if the protein includes multiple polypeptide chains, each chain can be cloned into an expression vector, e.g., the same or different vectors, that are expressed in the same or different cells.

Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibodies can also be produced in eukaryotic cells. In one embodiment, the antibodies (e.g., scFv's) are expressed in a yeast cell such as Pichia (see, e.g., Powers et al., 2001, J. Immunol. Methods. 251:123-35), Hanseula, or Saccharomyces.

In one preferred embodiment, antibodies are produced in mammalian cells. Preferred mammalian host cells for expressing the clone antibodies or antigen-binding fragments thereof include Chinese Hamster Ovary (CHO cells) (including dhfr-CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G coupled matrix.

For antibodies that include an Fc domain, the antibody production system may produce antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcg receptors and complement C1q (Burton and Woof, 1992, Adv. Immunol. 51:1-84; Jefferis et al., 1998, Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

One method for producing a transgenic mouse is as follows. Briefly, a targeting construct that encodes the antibody is microinjected into the male pronucleus of fertilized oocytes. The oocytes are injected into the uterus of a pseudopregnant foster mother for the development into viable pups. Some offspring incorporate the transgene.

Pharmaceutical Compositions Comprising Anti-Idiotypic Antibodies Against Anti-MMP-14 Antibodies

In another aspect, the disclosure provides compositions, e.g., pharmaceutically acceptable compositions or pharmaceutical compositions, which include an anti-idiotypic antibody against an anti-MMP-14 antibody described herein. The anti-idiotypic antibody can be formulated together with a pharmaceutically acceptable carrier. Pharmaceutical compositions include therapeutic compositions and diagnostic compositions, e.g., compositions that include labeled anti-idiotypic antibodies against an anti-MMP-14 antibody for in vivo imaging.

A pharmaceutically acceptable carrier includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal, or epidermal administration (e.g., by injection or infusion). Depending on the route of administration, the anti-idiotypic antibody may be coated in a material to protect the compound from the action of acids and other natural conditions that may inactivate the compound.

A pharmaceutically acceptable salt is a salt that retains the desired biological activity of the parent compound and does not impart any undesired toxicological effects (see e.g., Berge, S. M., et al., 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like.

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Many compositions are in the form of injectable or infusible solutions, such as compositions similar to those used for administration of humans with antibodies. An exemplary mode of administration is parenteral (e.g., intravenous, subcutaneous, intraperitoneal, intramuscular). In one embodiment, the anti-idiotypic antibody is administered by intravenous infusion or injection. In another preferred embodiment, the anti-idiotypic antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An anti-idiotypic antibody can be administered by a variety of methods known in the art, although for many applications, the preferred route/mode of administration is intravenous injection or infusion. For example, for therapeutic applications, the anti-idiotypic antibody can be administered by intravenous infusion at a rate of less than 30, 20, 10, 5, or 1 mg/min to reach a dose of about 1 to 100 mg/m2 or 7 to 25 mg/m2. The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., 1978, Marcel Dekker, Inc., New York.

In certain embodiments, the ligand may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound disclosed herein by other than parenteral administration, it may be necessary to coat the compound with, or co-administer the compound with, a material to prevent its inactivation.

Pharmaceutical compositions can be administered with medical devices known in the art. For example, in one embodiment, a pharmaceutical composition disclosed herein can be administered with a device, e.g., a needleless hypodermic injection device, a pump, or implant.

In certain embodiments, an anti-idiotypic antibody can be formulated to ensure proper distribution in vivo. For example, the blood-brain barrier (BBB) excludes many highly hydrophilic compounds. To ensure that the therapeutic compounds disclosed herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade, 1989, J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an anti-idiotypic antibody disclosed herein is 0.1-20 mg/kg, more preferably 1-10 mg/kg. An anti-idiotypic antibody can be administered, e.g., by intravenous infusion, e.g., at a rate of less than 30, 20, 10, 5, or 1 mg/min to reach a dose of about 1 to 100 mg/m2 or about 5 to 30 mg/m2. For ligands smaller in molecular weight than an antibody, appropriate amounts can be proportionally less. Dosage values may vary with the type and severity of the condition to be alleviated. For a particular subject, specific dosage regimens can be adjusted over time according to the individual need and the professional judgment of the person administering or supervising the administration of the compositions.

The pharmaceutical compositions disclosed herein may include a “therapeutically effective amount” or a “prophylactically effective amount” of an anti-idiotypic antibody disclosed herein. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition is outweighed by the therapeutically beneficial effects.

A “therapeutically effective dosage” preferably modulates a measurable parameter, e.g., levels of circulating IgG antibodies by a statistically significant degree or at least about 20%, more preferably by at least about 40%, even more preferably by at least about 60%, and still more preferably by at least about 80% relative to untreated subjects. The ability of a compound to modulate a measurable parameter, e.g., autoimmunity, can be evaluated in an animal model system predictive of efficacy in human autoimmune disorders. Alternatively, this property of a composition can be evaluated by examining the ability of the compound to modulate a parameter in vitro, e.g., by assays known to the skilled practitioner.

A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

Stabilization and Retention

In one embodiment, an anti-idiotypic antibody is physically associated with a moiety that improves its stabilization and/or retention in circulation, e.g., in blood, serum, lymph, or other tissues, e.g., by at least 1.5, 2, 5, 10, or 50 fold. For example, an anti-idiotypic antibody can be associated with a polymer, e.g., a substantially non-antigenic polymers, such as polyalkylene oxides or polyethylene oxides. Suitable polymers will vary substantially by weight. Polymers having molecular number average weights ranging from about 200 to about 35,000 (or about 1,000 to about 15,000, and 2,000 to about 12,500) can be used. For example, an anti-idiotypic antibody can be conjugated to a water soluble polymer, e.g., hydrophilic polyvinyl polymers, e.g. polyvinylalcohol and polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

Vaccines

The invention also provides a vaccine composition comprising an effective immunizing amount of the anti-idiotypic antibody and a pharmaceutically acceptable carrier.

The vaccine may also comprise a suitable medium. Suitable media include pharmaceutically acceptable carriers, such as phosphate buffered saline solution, liposomes and emulsions.

The vaccine may further comprise pharmaceutically acceptable adjuvants that may enhance the immune response, such as muramyl peptides, lymphokines, such as interferon, interleukin-1 and interleukin-6, or bacterial adjuvants. The adjuvant may comprise suitable particles onto which the anti-idiotypic antibody is adsorbed, such as aluminum oxide particles. These vaccine compositions containing adjuvants may be prepared as is known in the art. An example of a bacterial adjuvant is BCG.

Treatments Incorporating Pharmaceutical Compositions Comprising Anti-Idiotypic Antibodies Against Anti-MMP-14 Antibodies

Anti-idiotypic antibodies against anti-MMP-14 antibodies detailed herein have therapeutic and prophylactic utilities. These antibodies can be administered to a subject to reduce or even eliminate levels of circulating therapeutic anti-MMP-14 antibodies such as DX-2400.

The term “treating” refers to administering a therapy in an amount, manner, and/or mode effective to improve a condition, symptom, or parameter associated with a disorder or to prevent progression of a disorder, to either a statistically significant degree or to a degree detectable to one skilled in the art. An effective amount, manner, or mode can vary depending on the subject and may be tailored to the subject. The subject can be a human or a non-human animal, e.g., a non-human mammal.

The anti-idiotypic antibody can be administered in a therapeutically effective amount, e.g., such that upon single or multiple dose administration to a subject, the subject exhibits an amelioration of symptoms of a disorder, e.g., therapeutic antibody poisioning, of a parameter indicative of presence or risk for the disorder, or of toxic side effects of a therapeutic antibody or fusion IgG.

Therapeutic antibodies and IgG fusion proteins against whose toxicity the anti-idiotypic antibodies disclosed herein may serve as an antidote include, but are not limited to, DX-2400.

Methods of administering the anti-idiotypic antibodies are described in “Pharmaceutical Compositions.” Suitable dosages of the molecules used will depend on the age and weight of the subject and the particular drug used.

Where a vaccine composition is used in a method of vaccinating, the vaccine is presented to the immune system of the mammal in a form that induces an effective immune response, preferably combined with a pharmaceutically acceptable adjuvant.

The vaccine may be administered to a mammal by methods known in the art. Such methods include, for example, intravenous, intraperitoneal, subcutaneous, intramuscular, topical, or intradermal administration.

Diagnostic Compositions Comprising and Uses of Anti-Idiotypic Antibodies Against Anti-MMP-14 Antibodies

The anti-idiotypic antibodies against anti-MMP-14 antibodies identified by the method described herein and/or detailed herein have in vitro and in vivo diagnostic utilities.

In one aspect, the disclosure provides a diagnostic method for detecting the presence of an anti-MMP-14 antibody, in vitro or in vivo (e.g., in vivo imaging in a subject). The method can include localizing the anti-MMP-14 antibody to a subcellular location, e.g., the endosome. The method can include: (i) contacting a biological sample with an anti-idiotypic antibody specific for the anti-MMP-14 antibody; and (ii) detecting formation of a complex between the anti-idiotypic antibody and the biological sample. The method can also include contacting a reference sample (e.g., a control sample) with the ligand, and determining the extent of formation of the complex between the ligand and the sample relative to the same for the reference sample. A change, e.g., a statistically significant change, in the formation of the complex in the sample or subject relative to the control sample or subject can be indicative of the presence of anti-MMP-14 antibody in the sample. Another exemplary method includes: (i) administering the anti-idiotypic antibody specific for the anti-MMP-14 antibody to a subject; and (iii) detecting formation of a complex between the anti-idiotypic antibody specific for the anti-MMP-14 antibody and the subject. The detecting can include determining location or time of formation of the complex.

The anti-idiotypic antibody specific for an anti-MMP-14 antibody can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the anti-idiotypic antibody specific for an anti-MMP-14 antibody and the anti-MMP-14 antibody can be detected by measuring or visualizing either the ligand bound to the anti-MMP-14 antibody or unbound ligand. Conventional detection assays can be used, e.g., an enzyme-linked immunosorbent assays (ELISA), a radioimmunoassay (RIA) or tissue immunohistochemistry. Further to labeling the anti-idiotypic antibody specific for the anti-MMP-14 antibody, the presence of anti-MMP-14 antibody can be assayed in a sample by a competition immunoassay utilizing standards labeled with a detectable substance and an unlabeled anti-idiotypic antibody specific for the anti-MMP-14 antibody. In one example of this assay, the biological sample, the labeled standards, and the anti-idiotypic antibody specific for the anti-MMP-14 antibody are combined and the amount of labeled standard bound to the unlabeled ligand is determined. The amount of anti-MMP-14 antibody in the sample is inversely proportional to the amount of labeled standard bound to the anti-idiotypic antibody specific for the anti-MMP-14 antibody.

Fluorophore and chromophore labeled anti-idiotypic antibodies can be prepared. Because antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer, 1968, Science 162:526 and Brand, L. et al., 1972, Annu. Rev. Biochem. 41:843 868. The anti-idiotypic antibodies can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the anti-idiotypic antibody specific for an anti-MMP-14 antibody can be used to detect the presence or localization of the anti-MMP-14 antibody in a sample, e.g., using fluorescent microscopy (such as confocal or deconvolution microscopy).

Histological Analysis. Immunohistochemistry can be performed using the anti-idiotypic antibodies described herein. For example, the antibody can be synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation for binding, the preparation is washed to remove unbound antibody. The preparation is then analyzed, e.g., using microscopy, to identify if the antibody bound to the preparation. Of course, the antibody (or other polypeptide or peptide) can be unlabeled at the time of binding. After binding and washing, the antibody is labeled in order to render it detectable.

Protein Arrays. Anti-idiotypic antibodies specific for an anti-MMP-14 antibody can also be immobilized on a protein array. The protein array can be used as a diagnostic tool, e.g., to screen medical samples (such as isolated cells, blood, sera, biopsies, and the like). Of course, the protein array can also include other ligands, e.g., that bind to anti-MMP-14 antibody or to other target molecules.

Methods of producing polypeptide arrays are described, e.g., in De Wildt et al., 2000, Nat. Biotechnol. 18:989-994; Lueking et al., 1999, Anal. Biochem. 270:103-111; Ge, 2000, Nucleic Acids Res. 28, e3, I-VII; MacBeath and Schreiber, 2000, Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer. For example, the array can be an array of antibodies, e.g., as described in De Wildt, supra. Cells that produce the protein ligands can be grown on a filter in an arrayed format. Polypeptide production is induced, and the expressed polypeptides are immobilized to the filter at the location of the cell. A protein array can be contacted with a labeled target to determine the extent of binding of the target to each immobilized polypeptide. Information about the extent of binding at each address of the array can be stored as a profile, e.g., in a computer database. The protein array can be produced in replicates and used to compare binding profiles, e.g., of a target and a non-target.

FACS (Fluorescence Activated Cell Sorting). The anti-idiotypic antibodies specific for an anti-MMP-14 antibody can be used to label cells, e.g., cells in a sample (e.g., a patient sample). The ligand is also attached (or attachable) to a fluorescent compound. The cells can then be sorted using fluorescence activated cell sorter (e.g., using a sorter available from Becton Dickinson Immunocytometry Systems, San Jose Calif.; see also U.S. Pat. Nos. 5,627,037; 5,030,002; and 5,137,809). As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample. The sorter can also deflect the cell and separate cells bound by the ligand from those cells not bound by the ligand. The separated cells can be cultured and/or characterized.

In vivo Imaging. Also featured is a method for detecting the presence of tissues containing anti-MMP-14 antibodies in vivo. The method includes (i) administering to a subject (e.g., a patient being treated with an anti-MMP-14 antibody) an anti-idiotypic antibody specific for the anti-MMP-14 antibody, conjugated to a detectable marker; (ii) exposing the subject to a means for detecting said detectable marker to the anti-MMP-14 antibody-containing tissues or cells. For example, the subject is imaged, e.g., by NMR or other tomographic means.

Examples of labels useful for diagnostic imaging include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short range radiation emitters, such as isotopes detectable by short range detector probes can also be employed. The protein ligand can be labeled with such reagents using known techniques. For example, see Wensel and Meares, 1983, Radioimmunoimaging and Radioimmunotherapy, Elsevier, New York for techniques relating to the radiolabeling of antibodies and D. Colcher et al., 1986, Meth. Enzymol. 121: 802 816.

A radiolabeled anti-idiotypic antibody can also be used for in vitro diagnostic tests. The specific activity of a isotopically-labeled ligand depends upon the half life, the isotopic purity of the radioactive label, and how the label is incorporated into the antibody.

Procedures for labeling polypeptides with the radioactive isotopes (such as ¹⁴C, ³H, ³⁵S, ¹²⁵I, ³²P, ¹³¹I) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, J. W. (Monoclonal antibodies: principles and practice: production and application of monoclonal antibodies in cell biology, biochemistry, and immunology 2nd ed. London; Orlando: Academic Press, 1986. pp 124 126) and the references cited therein. Other procedures for iodinating polypeptides, such as antibodies, are described by Hunter and Greenwood, 1962, Nature 144:945, David et al., 1974, Biochemistry 13:1014 1021, and U.S. Pat. Nos. 3,867,517 and 4,376,110. Radiolabeling elements which are useful in imaging include ¹²³I, ¹³¹I, ¹¹¹In, and ^{99m}Tc, for example. Procedures for iodinating antibodies are described by Greenwood, F. et al., 1963, Biochem. J. 89:114 123; Marchalonis, J., 1969, Biochem. J. 113:299 305; and Morrison, M. et al., 1971, Immunochemistry 289 297. Procedures for ^{99m}Tc labeling are described by Rhodes, B. et al. in Burchiel, S. et al. (eds.), Tumor Imaging: The Radioimmunochemical Detection of Cancer, New York: Masson 111 123 (1982) and the references cited therein. Procedures suitable for ¹¹¹In labeling antibodies are described by Hnatowich, D. J. et al., 1983, J. Immunol. Methods, 65:147 157, Hnatowich, D. et al., 1984, J. Applied Radiation, 35:554 557, and Buckley, R. G. et al., 1984, F.E.B.S. 166:202 204.

In the case of a radiolabeled ligand, the ligand is administered to the patient, is localized to cells bearing the antigen with which the ligand reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65 85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 mm to about 10 nM in diameter). Particles can have ferromagnetic, antiferromagnetic, or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like.

The anti-idiotypic antibody specific for the anti-MMP-14 antibody can also be labeled with an indicating group containing of the NMR active ¹⁹F atom, or a plurality of such atoms inasmuch as (i) substantially all of naturally abundant fluorine atoms are the ¹⁹F isotope and, thus, substantially all fluorine containing compounds are NMR active; (ii) many chemically active polyfluorinated compounds such as trifluoracetic anhydride are commercially available at relatively low cost; and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett, 1982, Sci. Am. 246:78 88 to locate and image tissues containing anti-MMP-14 antibody.

Immunoassays

Provided also are immunoassays which detect or quantitate anti-MMP-14 antibodies, in a biological sample. An immunoassay for anti-MMP-14 antibody typically comprises incubating a biological sample in the presence of a detectably labeled high affinity anti-idiotypic antibody specific for the anti-MMP-14 antibody of the present invention capable of selectively binding to the anti-MMP-14 antibody, and detecting the labeled peptide or antibody which is bound in a biological sample. Various clinical assay procedures are well known in the art, e.g., as described in Immunoassays for the 80's, A. Voller et al., eds., University Park, 1981.

Thus, an anti-idiotypic antibody specific for an anti-MMP-14 antibody, can be added to nitrocellulose, or other solid support which is capable of immobilizing cells, cell particles or soluble proteins. The support can then be washed with suitable buffers followed by treatment with the detectably labeled anti-idiotypic antibody specific for an anti-MMP-14 antibody. The solid phase support can then be washed with the buffer a second time to remove unbound peptide or antibody. The amount of bound label on the solid support can then be detected by known method steps.

By “solid phase support” or “carrier” is intended any support capable of binding peptide, antigen or antibody. Well-known supports or carriers, include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to anti-MMP-14 or an anti-idiotypic antibody specific for the anti-MMP-14 antibody. Thus, the support configuration can be spherical, as in a bead, or cylindrical, as in the inside surface of a test tube, or the external surface of a rod. Alternatively, the surface can be flat such as a sheet, culture dish, test strip, etc. Preferred supports include polystyrene beads. Those skilled in the art will know many other suitable carriers for binding antibody, peptide or antigen, or can ascertain the same by routine experimentation.

Well known method steps can determine binding activity of a given lot of anti-idiotypic antibody specific for an anti-MMP-14 antibody. Those skilled in the art can determine operative and optimal assay conditions by routine experimentation.

Detectably labeling an anti-idiotypic antibody specific for the anti-MMP-14 antibody can be accomplished by linking to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the anti-idiotypic antibodies specific for an anti-MMP-14 antibody of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

By radioactively labeling the anti-idiotypic antibodies specific for an anti-MMP-14 antibody, it is possible to detect anti-MMP-14 antibody through the use of a radioimmunoassay (RIA) (see, for example, Work, et al., Laboratory Techniques and Biochemistry in Molecular Biology, North Holland Publishing Company, N.Y. (1978)). The radio-active isotope can be detected by such means as the use of a gamma counter or a scintillation counter or by autoradiography. Isotopes which are particularly useful for the purpose of the present invention are: ³H, ¹²⁵I, ¹³¹In, ³⁵S, ¹⁴C, and, preferably, ¹²⁵I. Radiolabeling is further described above.

It is also possible to label the anti-idiotypic antibodies with a fluorescent compound, as previously described and as described here. When the fluorescent labeled antibody is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine. The anti-idiotypic antibodies can also be detectably labeled using fluorescence-emitting metals such as ¹⁵²Eu, or others of the lanthanide series. These metals can be attached to the anti-idiotypic antibody using such metal chelating groups as diethylenetriaminepentaacetic acid (DTPA) or ethylenediamine-tetraacetic acid (EDTA).

The anti-idiotypic antibodies also can be detectably labeled by coupling to a chemiluminescent compound. The presence of the chemiluminescently labeled antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the anti-idiotypic antibody, fragment or derivative of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the anti-idiotypic antibody, fragment or derivative can be accomplished by a scintillation counter, for example, if the detectable label is a radioactive gamma emitter, or by a fluorometer, for example, if the label is a fluorescent material. In the case of an enzyme label, the detection can be accomplished by colorometric methods which employ a substrate for the enzyme. Detection can also be accomplished by visual comparison of the extent of enzymatic reaction of a substrate in comparison with similarly prepared standards.

For the purposes of the present invention, the anti-MMP-14 antibody which is detected by the above assays can be present in a biological sample, as discussed above and further discussed here. Any sample containing anti-MMP-14 antibody can be used. Preferably, the sample is a biological fluid such as, for example, blood, serum, lymph, urine, inflammatory exudate, cerebrospinal fluid, amniotic fluid, a tissue extract or homogenate, and the like. However, the invention is not limited to assays using only these samples, it being possible for one of ordinary skill in the art to determine suitable conditions which allow the use of other samples.

In situ detection can be accomplished by removing a histological specimen from a patient, and providing the combination of labeled antibodies of the present invention to such a specimen.

The antibody, fragment or derivative of the present invention can be adapted for utilization in an immunometric assay, also known as a “two-site” or “sandwich” assay. In a typical immunometric assay, a quantity of unlabeled antibody (or fragment of antibody) is bound to a solid support that is insoluble in the fluid being tested and a quantity of detectably labeled soluble antibody is added to permit detection and/or quantitation of the ternary complex formed between solid-phase antibody, antigen, and labeled antibody.

Typical, and preferred, immunometric assays include “forward” assays in which the antibody bound to the solid phase is first contacted with the sample being tested to extract the anti-MMP-14 antibody from the sample by formation of a binary solid phase anti-idiotypic antibody-anti-MMP-14 antibody complex. After a suitable incubation period, the solid support is washed to remove the residue of the fluid sample, including unreacted anti-MMP-14 antibody, if any, and then contacted with the solution containing a known quantity of labeled antibody (which functions as a “reporter molecule”). After a second incubation period to permit the labeled antibody to complex with the anti-MMP-14 antibody bound to the solid support through the unlabeled antibody, the solid support is washed a second time to remove the unreacted labeled antibody. This type of forward sandwich assay can be a simple “yes/no” assay to determine whether anti-MMP-14 antibody is present or can be made quantitative by comparing the measure of labeled antibody with that obtained for a standard sample containing known quantities of anti-MMP-14 antibody. Such “two-site” or “sandwich” assays are described by Wide (Radioimmune Assay Method, Kirkham, ed., Livingstone, Edinburgh, 1970, pp. 199-206).

Other types of “sandwich” assays are the so-called “simultaneous” and “reverse” assays. A simultaneous assay involves a single incubation step wherein the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period, is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays. In one embodiment, a combination of antibodies of the present invention specific for separate epitopes can be used to construct a sensitive three-site immunoradiometric assay.

Removal of Anti-MMP-14 Antibodies From Solutions or Samples Using Anti-Idiotypic Antibodies Against Anti-MMP-14 Antibodies

The anti-idiotypic antibodies against anti-MMP-14 antibodies of this invention, e.g., attached to a solid support, can be used to remove anti-MMP-14 antibodies from fluids or tissue or cell extracts. In a preferred embodiment, they are used to remove anti-MMP-14 antibodies from blood or blood plasma products. In another preferred embodiment, the anti-idiotypic antibodies are advantageously used in extracorporeal immunoadsorbent devices, which are known in the art (see, for example, Seminars in Hematology, 26 (2 Suppl. 1) (1989)). For example, patient blood or other body fluid is exposed to the attached antibody, resulting in partial or complete removal of circulating anti-MMP-14 antibody (free or in immune complexes), following which the fluid is returned to the body. This immunoadsorption can be implemented in a continuous flow arrangement, with or without interposing a cell centrifugation step. See, for example, Terman, et al., J. Immunol. 117:1971-1975 (1976).

The anti-idiotypic antibodies may also be used in standard biochemical purification protocols as is known to one of skill in the art such as affinity purification, for use in isolating and purifying anti-MMP-14 antibodies for pharmaceutical, reagent and other compositions and uses.

Kits

The present invention provides kits for practice of the afore-described methods. In certain embodiments, kits may comprise anti-idiotypic antibodies specific for an anti-MMP-14 antibody and optionally informational material. The informational material can be descriptive, instructional, marketing or other material that relates to the methods described herein and/or the use of an anti-idiotypic antibody specific for an anti-MMP-14 antibody for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to using the ligand for immunoabsorption, e.g., to treat, prevent, or diagnose a disorder described herein, e.g., therapeutic antibody poisoning. In one embodiment, the informational material can include instructions to administer an anti-idiotypic antibody in a suitable manner to perform the methods described herein, e.g., in a suitable dose, dosage form, or mode of administration (e.g., a dose, dosage form, or mode of administration described herein). In another embodiment, the informational material can include instructions to administer an anti-idiotypic antibody to a suitable subject, e.g., a human, e.g., a human having, or at risk for, an autoimmune disorder (e.g., rheumatoid arthritis or systemic lupus erythematosis).

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. In another embodiment, the informational material of the kit is contact information, e.g., a physical address, email address, website, or telephone number, where a user of the kit can obtain substantive information about an anti-idiotypic antibody and/or its use in the methods described herein. Of course, the informational material can also be provided in any combination of formats.

In addition to an anti-idiotypic antibody, the composition of the kit can include other ingredients, such as a solvent or buffer, a stabilizer, a preservative, a flavoring agent (e.g., a bitter antagonist or a sweetener), a fragrance or other cosmetic ingredient. Alternatively, the other ingredients can be included in the kit, but in different compositions or containers than an anti-idiotypic antibody. In such embodiments, the kit can include instructions for admixing an anti-idiotypic antibody and the other ingredients, or for using a an anti-idiotypic antibody together with the other ingredients.

An anti-idiotypic antibody can be provided in any form, e.g., liquid, dried or lyophilized form. It is preferred that an anti-idiotypic antibody be substantially pure and/or sterile when it is comprised within a pharmaceutical composition. When an anti-idiotypic antibody is provided in a liquid solution, the liquid solution preferably is an aqueous solution, with a sterile aqueous solution being preferred. When an anti-idiotypic antibody is provided as a dried form, reconstitution generally is by the addition of a suitable solvent. The solvent, e.g., sterile water or buffer, can optionally be provided in the kit.

Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, purification, and other applications.

Immunoadsorption

In some embodiments, the invention provides methods for the removal of an unwanted therapeutic antibody from an individual. In some embodiments, the unwanted therapeutic antibody is an anti-MMP14 antibody (e.g., DX-2400).

In some embodiments, the treatment methods presented herein may be combined with methods to remove or partially remove therapeutic antibodies from the bloodstream of a subject. In some embodiments, the anti-idiotype antibodies presented herein may be combined with a capture protein that can bind a therapeutic antibody, the combination resulting in an increased clearance of the therapeutic antibody from the bloodstream. In some embodiments, the method of removal or partial removal of the therapeutic antibody from the bloodstream of a subject is plasma exchange (PLEX). In some embodiments, the anti-idiotype antibodies can be administered to a subject undergoing plasma exchange. In some embodiments, the anti-idiotype antibodies can be used as an immunoadsorbant for an anti-MMP14 antibody in the plasma exchange process.

In plasma exchange (also called apheresis or plasmapheresis) blood is taken from the body and plasma containing an unwanted agent, such as a therapeutic antibody (e.g., an anti-MMP14 antibody), is removed from the blood by a cell separator. Blood can be removed from the body in batches or it can be removed in a continuous flow mode, with the latter allowing for the reintroduction of the processed blood into the body. The removed plasma comprising the unwanted agent will be discarded and the patient will receive donor plasma or saline with added proteins in return. In some embodiments, multiple rounds of plasma exchange may be needed to remove the unwanted agent from the blood or to lower the level of the unwanted agent in the blood to an acceptable level. In some embodiments the blood is “filtered” and the unwanted agent removed, before returning the blood to the patient. Methods of plasma exchange are well known in the art and are described for instance in U.S. Pat. No. 6,960,178.

Plasma exchange has been shown to reduce therapeutic antibody levels in the blood of a subject and the restoration of homeostasis (See e.g., Effect of plasma exchange in accelerating natalizumab clearance and restoring leukocyte function. Khatri et al; 2009; Neurology 72:402-409).

If the unwanted agent to be removed from the blood is an IgG based therapeutic antibody, this antibody can be removed by contacting the blood with the capture protein Staphylococcal protein A, which will bind the Fc region of IgG and remove the IgG antibody from the bloodstream. Other capture proteins can be used for different isotype antibodies. In some embodiments, the anti-idiotype antibodies can be used as a capture protein in the plasma exchange process. In some embodiments, the anti-idiotype antibodies are administered to the patient during or before plasma exchange. In some embodiments, the anti-idiotype antibodies can be immobilized and used in a column, resulting in the binding of an anti-MMP14 antibody, e.g., immmobilization of the anti-MMP14 antibody to the column. In some embodiments, the blood of a patient that has a therapeutic antibody can be contact both with immobilized anti-idiotype antibody and immobilized protein A.

In some embodiments the anti-idiotype antibodies presented herein can be used in “rescue” therapy for therapeutic antibodies that have been administered and have shown an adverse effect in a subject (e.g., therapeutic antibody poisoning). In some embodiments, the anti-idiotype antibodies can be used as an alternative for plasma exchange. The administration of anti-idiotype can accomplish therapeutic antibody depletion without the risks associated with plasmapheresis and plasma exchange such as vascular access, citrate therapy and donor plasma sourcing.

### EXAMPLES

The present invention is further illustrated by the following examples which should not be construed as limiting in any way. The contents of all references, pending patent applications and published patents, cited throughout this application are hereby expressly incorporated by reference.

### Example 1

Using the Dyax human phage displayed antibody library (FAB-310), we selected and screened for anti-idiotypic antibodies which bind specifically to the CDR regions of DX-2400. Among the 49 unique soluble Fabs that were tested for use in different serum matrices (Human, Cyno, Rat and Mouse), two anti-idiotypic DX-2400 Fabs were identified and used in developing a sensitive and drug-specific Meso Scale Discovery electrochemiluminescence assay method. The soluble Fabs reformatted as full IgGs could be used as surrogate positive control antibodies in developing immunogenicity and functional neutralizing antibody assays.

Using the Dyax phage displayed FAB-310 library, 49 anti-idiotypic Fabs displayed on phagemids were selected that potentially bound to the unique CDR sequences of DX-2400 IgG. Three rounds of selection were done against biotinylated DX-2400 after depleting the library using biotinylated-DX-2300 IgG (control antibody that binds unrelated target) and biotinylated matched Vk Fab (371L-X002-A03) (isotype matched control) immobilized on streptavidin magnetic beads. Approximately 1920 phage isolates were screened by phage ELISA for binding to DX-2400 and 192 primary ELISA positive Fab on phagemid were DNA sequenced. Forty-nine unique phage clones which had distinct heavy chains were secondarily screened by phage ELISA and ranked for specific binding to DX-2400. Later 45 soluble Fabs were expressed, purified and screened for non-specific binding to 5% human, cyno, rat, and mouse serum antibodies by using Fab as capture reagent and detecting with alkaline phosphatase-conjugated mouse anti-human Fc gamma-specific antibody. Two soluble Fab clones which showed specific binding to DX-2400 by phage ELISA and showed the least non-specific binding to endogenous serum antibodies from different serum matrices were selected for further development.

Master soluble anti-idiotypic Fab clones, 539C-M0016-E11 and 539C-M0021-E01 were each labeled with both biotin and ruthenium to be used as capture and detection pairing reagents for the electrochemiluminescent assay development. The resulting established electrochemiluminescent assay is very sensitive and specific. It is a sequential format assay in which biotinylated 539C-M0016-E11 anti-idiotypic Fab is captured to the streptavidin-coated plates and bound DX-2400 is detected by ruthenylated mouse anti-human Fc gamma-specific antibody.

In Table 3 are the amino-acid sequences of the light chain and heavy chain for 539C-M0016-E11 and 539C-M0021-E01. The light chains comprise a signal sequence, a VL, and Ckappa. The heavy chains comprise a signal sequence, VH and CH1. All DNA sequences that encode these amino-acid sequences are claimed.

TABLE 3
Amino-acid sequences

539C-M0016-E11
Light chain (LC)
MKKLLFAIPLVVPFYSHSAQDIQMTQSPATLSLSPGERATLSCRASQSVS
SYLAWYQQKPGQAPRLLIYDASNRATGIPARFSGSGSGTDFTLTISSLEP
EDFAVYYCQQRSNWPLTFGGGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC
Heavy Chain (HC)
MKKLLFAIPLVVPFVAQPAMAEVQLLESGGGLVQPGGSLRLSCAASGFTF
SFYVMDWVRQAPGKGLEWVSGIGPSGGSTDYADSVKGRFTISRDNSKNTL
YLQMNSLRAEDTAVYYCTRIRYDSSGYPTDAFDIWGQGTMVTVSSASTKG
PSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSC
539C-M0021-E01
Light Chain (LC)
MKKLLFAIPLVVPFYSHSAQDIQMTQSPSSLSASEGDRITLTCRASQSIS
IYLNWYQQKPGRAPKLLMYAATTLQSGVPSRFSGSGSGTDFTLTISGLRP
EDFATYYCQQSYDIPLTFGGGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC
Heavy Chain (HC)
MKKLLFAIPLVVPFVAQPAMAEVQLLESGGGLVQPGGSLRLSCAASGFTF
SVYPMIWVRQAPGKGLEWVSYISSSGGQTWYADSVKGRFTISRDNSKNTL
YLQMNSLRAEDTATYYCARGDDYDSSGPDYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSC

### Example 2

DX-2400: DX-2400 is an inhibitory MMP-14 binding antibody. The variable domain sequences for DX-2400 are:

| | |
|---|---|
| VH: | |
| DX-2400 | FR1--------------------------- CDR1- |
| | EVQLLESGGGLVQPGGSLRLSCAASGFTFS LYSMN |
| DX-2400 | FR2----------- CDR2------- CDR2-- |
| | WVRQAPGKGLEWVS SIYSSGGSTLY ADSVKG |
| DX-2400 | FR3----------------------------- CDR3-- |
| | RFTISRDNSKNTLYLQMNSLRAEDTAVYYCAR GRAFDI |
| DX-2400 | FR4--------- |
| | WGQGTMVTVSS |

CDR regions are in bold.

| | |
|---|---|
| VL: | |
| DX-2400 | FR1-------------------- CDR1------- |
| | DIQMTQSPSSLSASVGDRVTITC RASQSVGTYLN |
| DX-2400 | FR2------------ CDR2--- |
| | WYQQKPGKAPKLLIY ATSNLRS GVPS |
| DX-2400 | FR3------------------------- CDR3------ |
| | RFSGSGSGTDFTLTISSLQPEDFATYYC QQSYSIPRFT |
| DX-2400 | FR4------- |
| | FGPGTKVDIK |

CDR regions are in bold.

### Example 3

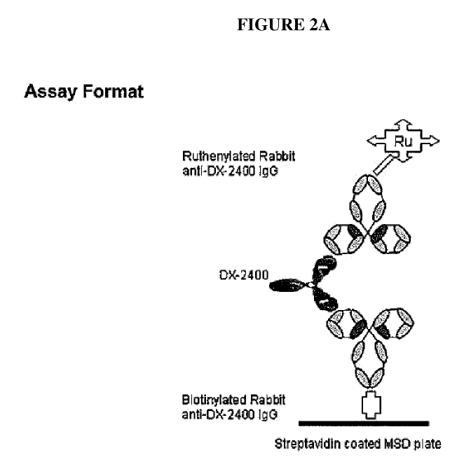

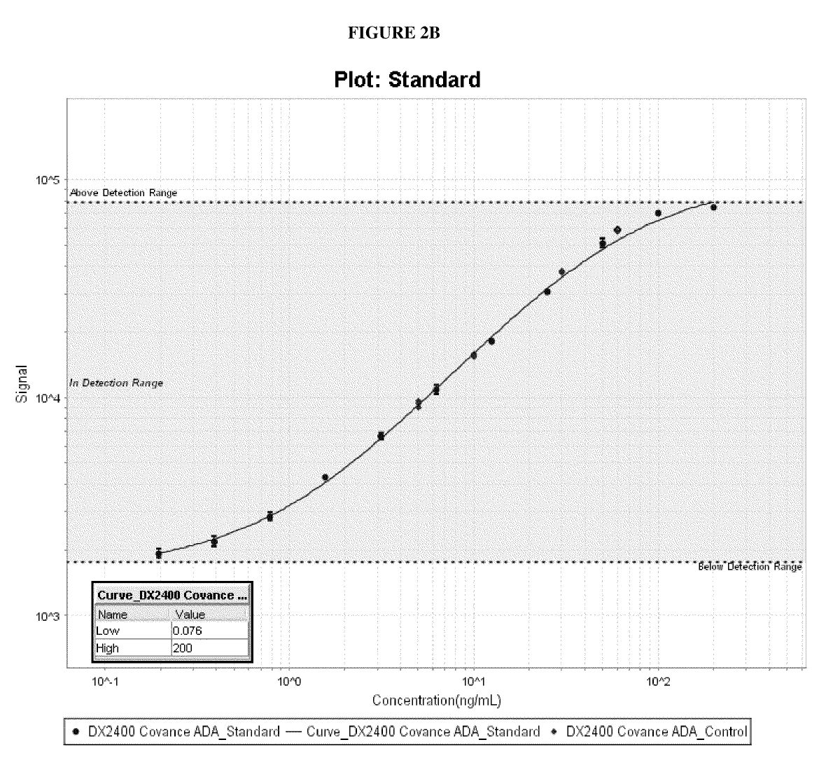

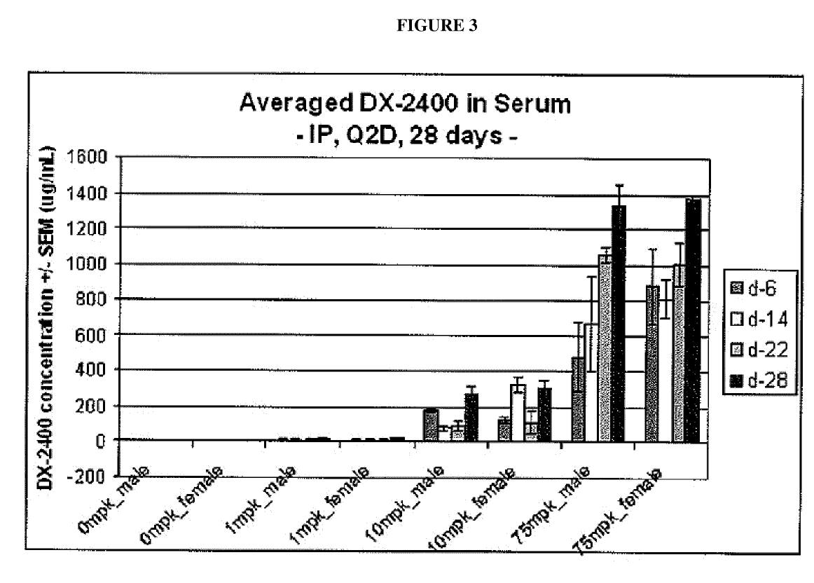

The DX-2400 pharmacokinetic assay for the drug exposure measurement utilizes an electrochemiluminescence (ECL) assay, similar in format to a standard ELISA assay. Biotinylated affinity purified rabbit anti DX-2400 antibodies are coated on a streptavidin plate. DX-2400 standards, controls, and samples are incubated on the plate and unbound reactants are washed off. The bound reactants are detected by incubating with Ruthenylated affinity purified rabbit anti DX-2400 antibodies. Captured Ruthenylated detector antibodies are read by means of an ECL imager, with the signal generated being proportional to the concentration of drug present in the sample. The actual concentration may be determined by interpolation from a standard curve. FIG. 2A shows the ECL assay format used and FIG. 2B shows a representative calibration curve-concentration in 5% rat serum. This ECL method was used to analyze rat samples from study CB07-5050-R-TX that were treated with DX-2400 at 0, 1, 10 and 75 mg/kg. The detected concentration of DX-2400 (FIG. 3) was calculated by interpolation from the calibration curve made with DX-2400 (FIG. 2B).

### Example 4

Anti-idiotypic Fabs for DX-2400 were generated by phage selection and screenings using DX-2400 as a target. Two thousand individual isolates were screened in high-throughput Fab-on-phage ELISA and a total of 192 binders were identified. Forty nine were distinct as determined by DNA sequencing. These 49 unique clones were screened as phages and sFabs (soluble Fabs) in 5% pooled normal serum matrices (human, mouse, rat and cynomolgus monkey). Top 10 unique clones were ranked according to the following criteria: strong binding to DX-2400, weak binding to DX-2300, low-non specific binding to streptavidin and Fab control.

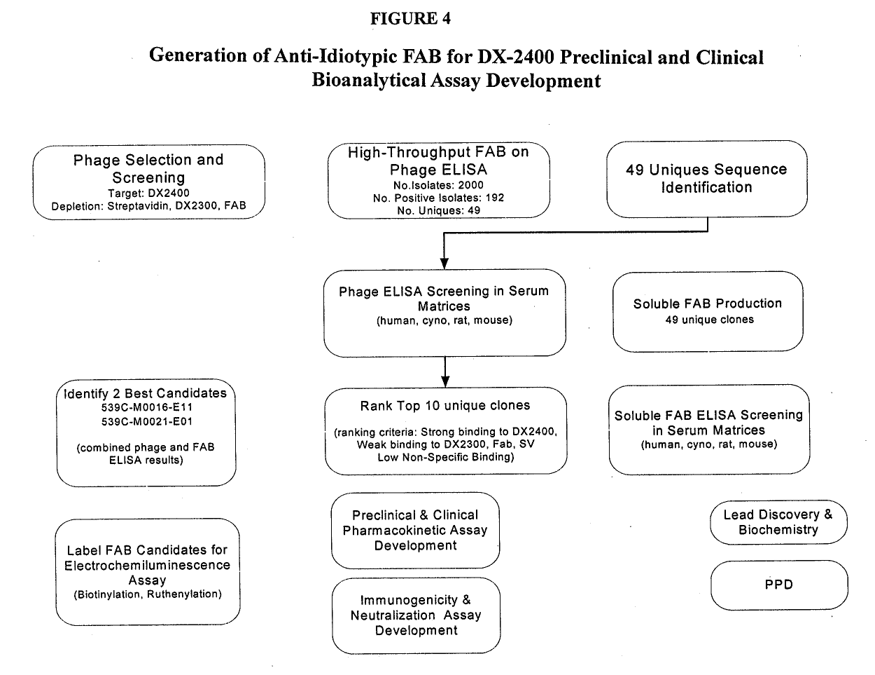

FIG. 4 summarizes the Bioanalytical Assay Development Flowchart.

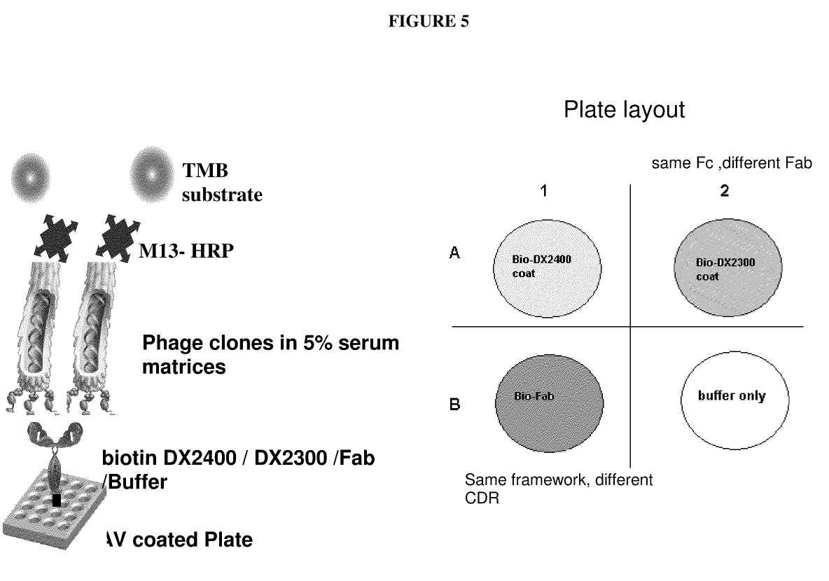

FIG. 5 shows the phage-ELISA screening.

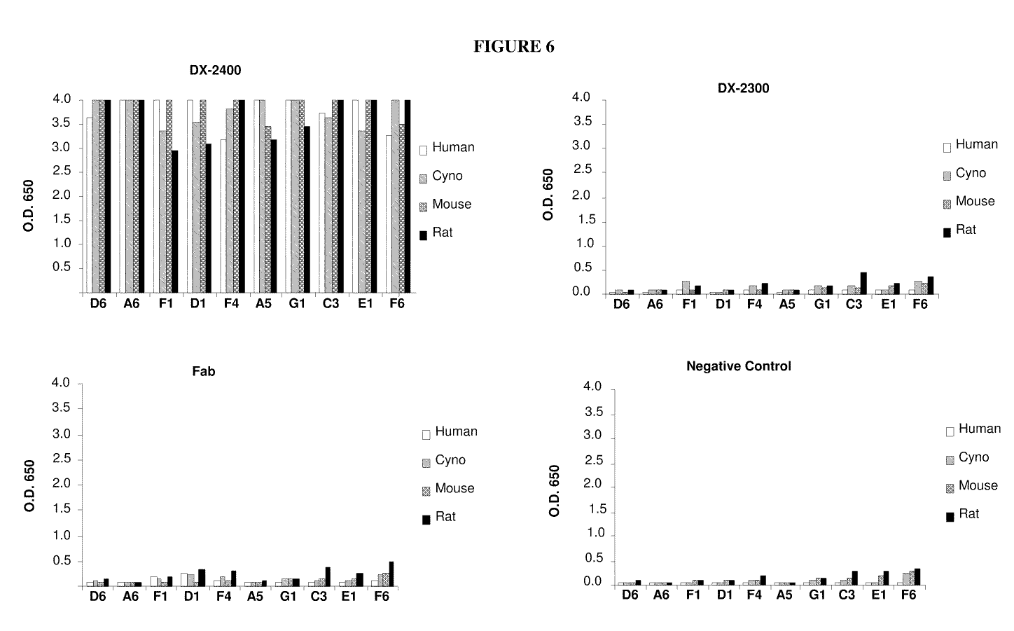

FIG. 6 shows the specificity assessment in the presence of 5% serum matrices (Phage ELISA).

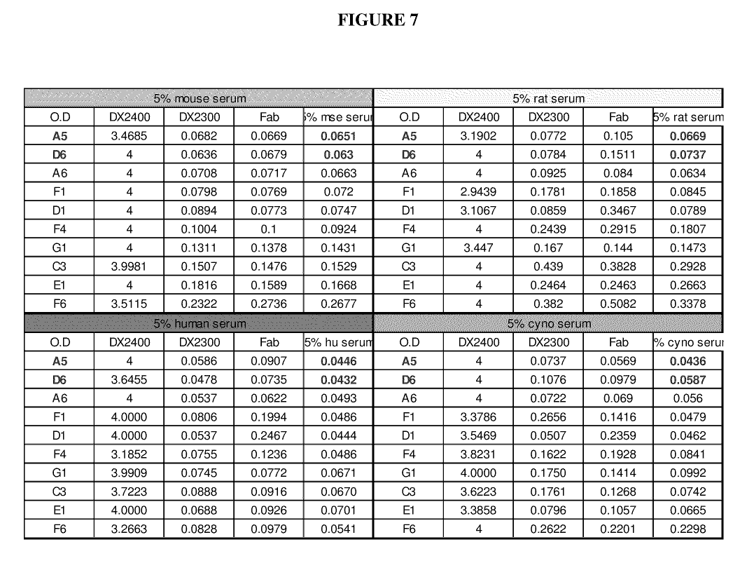

FIG. 7 shows the specificity ranking of phage candidates.

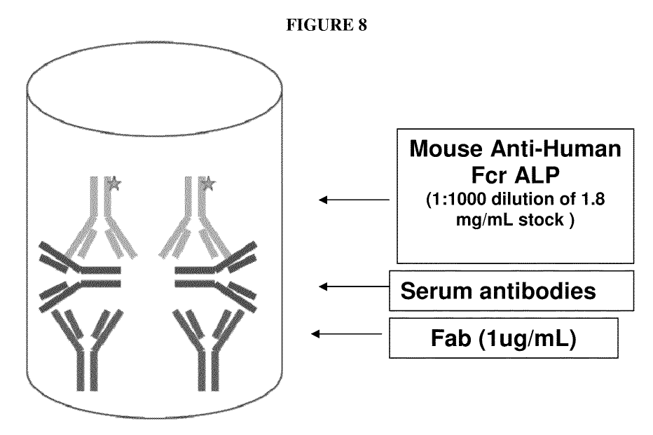

FIG. 8 shows the ELISA format used for anti-idiotype Fab screening.

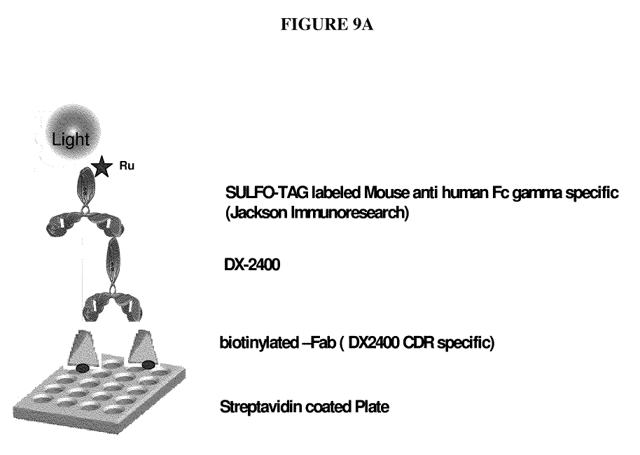

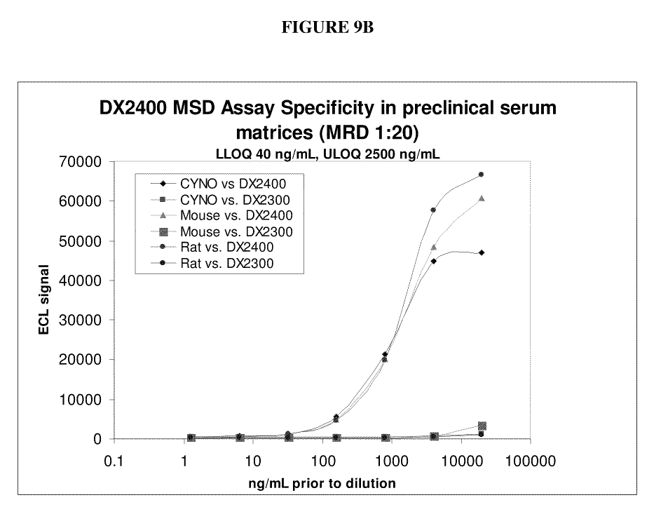

FIG. 9A shows the assay format used. FIG. 9B shows representative standard curves of DX-2400 and illustrates the assay specificity in preclinical serum matrices.

Ten anti-idiotypic Fabs to DX-2400 were selected as candidates for DX-2400 anti-idiotypic pharmacokinetic assay development. The final chosen anti-idiotypic Fab 539C-M0016-E11 was biotinylated as capture reagent. The DX-2400 standard curves were generated by serial titration of neat serum containing DX-2400.

The biotinylated 539C-M0016-E11 was coated on the streptavidin plate followed by incubation with DX-2400 standards. Unbound reactants were washed off, and the bound reactants were detected by Ruthenylated mouse anti-human Fc gamma specific antibodies.

### Example 5

The sequences of additional anti-idiotype antibodies to DX-2400 are as follows. These antibodies were isolated from screening a phage display antibody library (see above).

| Isolate | Initial Name | L-Info | H-Info | L-Leader | LV-FR1 |
|---|---|---|---|---|---|
| 539C-R0010-A01 | 539C-M0016-E11 | L. KU | H | FYSHSA | QDIQMTQSPATLSLSPGERATLSC |
| 539C-R0010-B01 | 539C-M0021-E01 | L. KU | H | FYSHSA | QDIQMTQSPSSLSASEGDRITLTC |
| 539C-R0010-B06 | 539C-M0012-C07 | L. KU | H | FYSHSA | QDIQMTQSPATLSVSPGENATLSC |
| 539C-R0010-C05 | 539C-M0010-D01 | L. KU | H | FYSHSA | QDIQMTQSPSSLSASVGDRVTITC |
| 539C-R0010-C06 | 539C-M0015-D11 | L. KU | H | FYSHSA | QDIQMTQSPSSLSASVGDRVTITC |
| 539C-R0010-D06 | 539C-M0006-D09 | L. LU | H | FYSHSA | QYELTQPHSVSESPGKTVTISC |
| 539C-R0010-E06 | 539C-M0014-D05 | L. LU | H | FYSHSA | QSALTQPASVSGSPGQSITISC |
| 539C-R0010-F05 | 539C-M0020-F11 | L. LU | H | FYSHSA | QSVLTQPPSVSVAPGQTATISC |
| 539C-R0010-F06 | 539C-M0016-F12 | L. KU | H | FYSHSA | QDIQMTQSPASLSASVGDRVTITC |
| 539C-R0004-G04 | 539C-M0008-D09 | L. LU | H | FYSHSA | QYELTQPPSVSVAPGQTARIIC |

| Isolate | Initial Name | LV-CDR1 | LV-FR2 | LV-CDR2 |
|---|---|---|---|---|
| 539C-R0010-A01 | 539C-M0016-E11 | RASQSVSSYLA | WYQQKPGQAPRLLIY | DASNRAT |
| 539C-R0010-B01 | 539C-M0021-E01 | RASQSISIYLN | WYQQKPGRAPKLLMY | AATTLQS |
| 539C-R0010-B06 | 539C-M0012-C07 | RASQSVASNLA | WYQQKPGQAPRLLIY | GASTRAT |
| 539C-R0010-C05 | 539C-M0010-D01 | LPSQDISVYLN | WYQQKPGEAPKLLIS | ATSDLQS |
| 539C-R0010-C06 | 539C-M0015-D11 | RASQNINTFLN | WYQQKPGKAPKLLIY | GASSLQS |
| 539C-R0010-D06 | 539C-M0006-D09 | TRSNGTIDNSFVQ | WYQQRPGSAPTTVIY | EDTQRPS |
| 539C-R0010-E06 | 539C-M0014-D05 | TGTSSDVGGYKLVS | WYQQHPGKVPKLIIS | EVSYRPS |
| 539C-R0010-F05 | 539C-M0020-F11 | EGDNIGSKSVH | WYQQRPGQAPVLVVY | DDYDRPS |
| 539C-R0010-F06 | 539C-M0016-F12 | QASRDIGEYLN | WYQQKAGKPPKLLIS | DATTLET |
| 539C-R0004-G04 | 539C-M0008-D09 | GGNDIGSKGVH | WYHQKAGQAPVLLVY | DNTDRPS |

| Isolate | Initial Name | LV-FR3 | LV-CDR3 | LV-FR4 | L-Constant |
|---|---|---|---|---|---|
| 539C-R0010-A01 | 539C-M0016-E11 | GIPARFSGSGSGTDFTLTISSLEPEDFAVYYC | QQRSNWPLT | FGGGTKVEIK | RTVAAPS |
| 539C-R0010-B01 | 539C-M0021-E01 | GVPSRFSGSGSGTDFTLTISGLRPEDFATYYC | QQSYDIPLT | FGGGTKVEIK | RTVAAPS |
| 539C-R0010-B06 | 539C-M0012-C07 | GAPARFSGSGSETDFTLTISSLQSEDFAVYYC | QQYHNWPPWT | FGQGTKVEIK | RTVAAPS |
| 539C-R0010-C05 | 539C-M0010-D01 | GVPSRFGGSGYGTDFSLTITSLQREDFATYYC | QQSYSLPFT | FGGGTRVEIK | RTVAAPS |
| 539C-R0010-C06 | 539C-M001S-D11 | GVPSRFSGSGSGTDFTLTISSLQPEDFATYYC | QQSSDALVYN | FGQGTKLEIK | RTVAAPS |
| 539C-R0010-D06 | 539C-M0006-D09 | GVPDRFSGSIDSSSNSASLTISGLKTEDEADYYC | QSYDSTNRGV | FGGGTKLTVL | GQPKAAP |
| 539C-R0010-E06 | 539C-M0014-D05 | GVSNRFSGSKSGNAASLTISGLQAEDEADYYC | SSYTSTSTWV | FGGGTKLTVL | GQPKAAP |
| 539C-R0010-F05 | 539C-M0020-F11 | GIPERFSGFNSGNTATLTIYRVEAGDEADFYC | QVRDSRTEERV | FGGGTKLTVL | GQPKAAP |
| 539C-R0010-F06 | 539C-M0016-F12 | GVPSRFSGTGSGTEFLFTISSVEPEDFATYYC | QQYDDLTWIS | FGPGTRLDVK | RTVAAPS |
| 539C-R0004-G04 | 539C-M0008-D09 | GIPERFSGSNSGNTAALTITRVEAGDEADYFC | QVWDSTGEHAV | FGGGTKLTVL | GQPKAAP |

| Isolate | Initial Name | H-Leader | HV-FR1 | HV-CDR1 |
|---|---|---|---|---|
| 539C-R0010-A01 | 539C-M0016-E11 | MKKLLFAIPLVVPFVAQPAMA | EVQLLESGGGLVQPGGSLRLSCAASGFTFS | FYVMD |
| 539C-R0010-B01 | 539C-M0021-E01 | MKKLLFAIPLVVPFVAQPASA | EVQLLESGGGLVQPGGSLRLSCAASGFTFS | VYPMI |
| 539C-R0010-B06 | 539C-M0012-C07 | MKKLLFAIPLVVPFVAQPASA | EVQLLESGGGLVQPGGSLRLSCAASGFTFS | EYQMN |
| 539C-R0010-C05 | 539C-M0010-D01 | MKKLLFAIPLVVPFVAQPASA | EVQLLESGGGLVQPGGSLRLSCAASGFTFS | GYGMG |
| 539C-R0010-C06 | 539C-M0015-D11 | MKKLLFAIPLVVPFVAQPASA | EVQLLESGGGLVQPGGSLRLSCAASGFTFS | SYNMA |
| 539C-R0010-D06 | 539C-M0006-D09 | MKKLLFAIPLVVPFVAQPASA | EVQLLESGGGLVQPGGSLRLSCAASGFTFS | DYSMY |
| 539C-R0010-E06 | 539C-M0014-D05 | MKKLLFAIPLVVPFVAQPASA | EVQLLESGGGLVQPGGSLRLSCAASGFTFS | DYWMF |
| 539C-R0010-F05 | 539C-M0020-F11 | MKKLLFAIPLVVPFVAQPASA | EVQLLESGGGLVQPGGSLRLSCAASGFTFS | WYAMV |
| 539C-R0010-F06 | 539C-M0016-F12 | MKKLLFAIPLVVPFVAQPAMA | EVQLLESGGGLVQPGGSLRLSCAASGFTFS | DYHMS |
| 539C-R0004-G04 | 539C-M0008-D09 | MKKLLFAIPLVVPFVAQPAMA | EVQLLESGGGLVQPGGSLRLSCAASGFTFS | DYDMH |

| Isolate | Initial Name | HV-FR2 | HV-CDR2 | HV-FR3 |
|---|---|---|---|---|
| 539C-R0010-A01 | 539C-M0016-E11 | WVRQAPGKGLEWVS | GIGPSGGSTDYADSVKG | RFTISRDNSKNTLYLQMNSLRAEDTAVYYCTR |
| 539C-R0010-B01 | 539C-M0021-E01 | WVRQAPGKGLEWVS | YISSSGGQTWYADSVKG | RFTISRDNSKNTLYLQMNSLRAEDTATYYCAR |
| 539C-R0010-B06 | 539C-M0012-C07 | WVRQAPGKGLEWVS | SIYSSGGGTAYADSVKG | RFTISRDNSKNTLYLQMNSLRAEDTAVYYCAK |
| 539C-R0010-C05 | 539C-M0010-D01 | WVRQAPGKGLEWVS | YIVPSGGATDYADSVKG | RFTISRDNSKNTLYLQMNSLRAEDTAVYYCAR |
| 539C-R0010-C06 | 539C-M0015-D11 | WVRQAPGKGLEWVS | VIVPSGGLTGYADSVKG | RFTISRDNSKNTLYLQMNSLRAEDTAVYYCAR |
| 539C-R0010-D06 | 539C-M0006-D09 | WVRQAPGKGLEWVS | YISSSGGNTEYADSVKG | RFTISRDNSKNTLYLQMNSLRAEDTAMYYCAT |
| 539C-R0010-E06 | 539C-M0014-D05 | WVRQAPGKGLEWVS | GIGPSGGIISYADSVKG | RFTISRDNSKNTLYLQMNSLRAEDTAVYYCAK |
| 539C-R0010-F05 | 539C-M0020-F11 | WVRQAPGKGLEWVS | GISPSGGLTSYADSVKG | RFTISRDNSKNTLYLQMNSLRAEDTAVYYCAR |
| 539C-R0010-F06 | 539C-M0016-F12 | WVRQAPGKGLEWVS | GISSSGGETFYADSVKG | RFTISRDNSKNTLYLQMNSLRAEDTAVYYCAS |
| 539C-R0004-G04 | 539C-M0008-D09 | WVRQAPGKGLEWVS | GISPSGGWTWYADSVKG | RFTISRDNSKNTLYLQMNSLRAEDTAVYYCAR |

| Isolate | Initial Name | HV-CDR3 | HV-FR4 | H-Constant |
|---|---|---|---|---|
| 539C-R0010-A01 | 539C-M0016-E11 | IRYDSSGYPTDAFDI | WGQGTMVTVSS | ASTKGPSVFPLAPSSKS |
| 539C-R0010-B01 | 539C-M0021-E01 | GDDYDSSGPDY | WGQGTLVTVSS | ASTKGPSVFPLAPSSKS |
| 539C-R0010-B06 | 539C-M0012-C07 | DQGDGYNYGVGLDY | WGQGTLVTVSS | ASTKGPSVFPLAPSSKS |
| 539C-R0010-C05 | 539C-M0010-D01 | AYYYDSSGFDI | WGQGTMVTVSS | ASTKGPSVFPLAPSSKS |
| 539C-R0010-C06 | 539C-M0015-D11 | ERGGFYSSGWYRYWFDP | WGQGTLVTVSS | ASTKGPSVFPLAPSSKS |
| 539C-R0010-D06 | 539C-M0006-D09 | TPSIWLGDLLENGPY | WGQGTLVTVSS | ASTKGPSVFPLAPSSKS |
| 539C-R0010-E06 | 539C-M0014-D05 | YGDYVSGFDY | WGQGTLVTVSS | ASTKGPSVFPLAPSSKS |
| 539C-R0010-F05 | 539C-M0020-F11 | KQMSPDYYYGMDV | WGQGTTVTVSS | ASTKGPSVFPLAPSSKS |
| 539C-R0010-F06 | 539C-M0016-F12 | GSHRDGYNLGYFDY | WGQGTLVTVSS | ASTKGPSVFPLAPSSKS |
| 539C-R0004-G04 | 539C-M0008-D09 | DWYSSGLDY | WGQGTLVTVSS | ASTKGPSVFPLAPSSKS |

| Isolate | Initial Name | LV-AA |
|---|---|---|
| 539C-R0010-A01 | 539C-M0016-E11 | QDIQMTQSPATLSLSPGERATLSCRASQSVSSYLAWYQQKPGQAPRLLIYDAS |
| | | NRATGIPARFSGSGSGTDFTLTISSLEPEDFAVYYCQQRSNWPLTFGGGTKVE |
| | | IK |
| 539C-R0010-B01 | 539C-M0021-E01 | QDIQMTQSPSSLSASEGDRITLTCRASQSISIYLNWYQQKPGRAPKLLMYAAT |
| | | TLQSGVPSRFSGSGSGTDFTLTISGLRPEDFATYYCQQSYDIPLTFGGGTKVE |
| | | IK |
| 539C-40010-B06 | 539C-M0012-C07 | QDIQMTQSPATLSVSPGENATLSCRASQSVASNLAWYQQKPGQAPRLLIYGAS |
| | | TRATGAPARFSGSGSETDFTLTISSLQSEDEAVYYCQQYHNWPPWTFGQGTKV |
| | | EIK |
| 539C-R0010-C05 | 539C-M0010-D01 | QDIQMTQSPSSLSASVGDRVTITCLPSQDISVYLNWYQQKPGEAPKLLISATS |
| | | DLQSGVPSRFGGSGYGTDFSLTITSLQREDFATYYCQQSYSLPFTFGGGTRVE |
| | | IK |
| 539C-R0010-C06 | 539C-M0015-D11 | QDIQMTQSPSSLSASVGDRVTITCRASQNINTFLNWYQQKPGKAPKLLIYGAS |
| | | SLQSGVPSRFSGSGSGTDFTLTISSLQPEDEATYYCQQSSDALVYNEGQGTKL |
| | | EIK |
| 539C-R0010-D06 | 539C-M0006-D09 | QYELTQPHSVSESPGKTVTISCTRSNGTIDNSFVQWYQQRPGSAPTTVIYEDT |
| | | QRPSGVPDRFSGSIDSSSNSASLTISGLKTEDEADYYCQSYDSTNRGVFGGGT |
| | | KLTVL |
| 539C-R0010-E06 | 539C-M0014-D05 | QSALTQPASVSGSPGQSITISCTGTSSDVGGYKLVSWYQQHPGKVPKLIISEV |
| | | SYRPSGVSNRFSGSKSGNAASLTISGLQAEDEADYYCSSYTSTSTWVFGGGTK |
| | | LTVL |
| 539C-R0010-F05 | 539C-M0020-F11 | QSVLTQPPSVSVAPGQTATISCEGDNIGSKSVHWYQQRPGQAPVLVVYDDYDR |
| | | PSGIPERFSGFNSGNTATLTIYRVEAGDEADFYCQVRDSRTEERVFGGGTKLT |
| | | VL |
| 539C-R0010-F06 | 539C-M0016-F12 | QDIQMTQSPASLSASVGDRVTITCQASRDIGEYLNWYQQKAGKPPKLLISDAT |
| | | TLETGVPSRFSGTGSGTEFLFTISSVEPEDFATYYCQQYDDLTWISFGPGTRL |
| | | DVK |
| 539C-R0004-G04 | 539C-M0008-D09 | QYELTQPPSVSVAPGQTARIICGGNDIGSKGVHWYHQKAGQAPVLLVYDNTDR |
| | | PSGIPERFSGSNSGNTAALTITRVEAGDEADYECQVWDSTGEHAVFGGGTKLT |
| | | VL |
| Isolate | Initial Name | HV-AA |
| 539C-R0010-A01 | 539C-M0016-E11 | EVQLLESGGGLVQPGGSLRLSCAASGFTFSFYVMDWVRQAPGKGLEWVSGIGP |
| | | SGGSTDYADSVKGRFTISRDNSKNTLYLQMNSLRAEDTAVYYCTRIRYDSSGY |
| | | PTDAFDIWGQGTMVTVSS |
| 539C-R0010-B01 | 539C-M0021-E01 | EVQLLESGGGLVQPGGSLRLSCAASGFTFSVYPMIWVRQAPGKGLEWVSYISS |
| | | SGGQTWYADSVKGRFTISRDNSKNTLYLQMNSLRAEDTATYYCARGDDYDSSG |
| | | PDYWGQGTLVTVSS |
| 539C-R0010-B06 | 539C-M0012-C07 | EVQLLESGGGLVQPGGSLRLSCAASGFTFSEYQMNWVRQAPGKGLEWVSSIYS |
| | | SGGGTAYADSVKGRFTISRDNSKNTLYLQMNSLRAEDTAVYYCAKDQGDGYNY |
| | | GVGLDYWGQGTLVTVSS |
| 539C-R0010-C05 | 539C-M0010-D01 | EVQLLESGGGLVQPGGSLRLSCAASGFTFSGYGMGWVRQAPGKGLEWVSYIVP |
| | | SGGATDYADSVKGRFTISRDNSKNTLYLQMNSLRAEDTAVYYCARAYYYDSSG |
| | | FDIWGQGTMVTVSS |
| 539C-R0010-C06 | 539C-M0015-D11 | EVQLLESGGGLVQPGGSLRLSCAASGFTFSSYNMAWVRQAPGKGLEWVSVIVP |
| | | SGGLTGYADSVKGRFTISRDNSKNTLYLQMNSLRAEDTAVYYCARERGGFYSS |
| | | GWYRYWFDPWGQGTLVTVSS |
| 539C-R0010-D06 | 539C-M0006-D09 | EVQLLESGGGLVQPGGSLRLSCAASGFTFSDYSMYWVRQAPGKGLEWVSYISS |
| | | SGGNTEYADSVKGRFTISRDNSKNTLYLQMNSLRAEDTAMYYCATTPSIWLGD |
| | | LLENGPYWGQGTLVTVSS |
| 539C-R0010-E06 | 539C-M0014-D05 | EVQLLESGGGLVQPGGSLRLSCAASGFTFSDYWMFWVRQAPGKGLEWVSGIGP |
| | | SGGIISYADSVKGRFTISRDNSKNTLYLQMNSLRAEDTAVYYCAKYGDYVSGF |
| | | DYWGQGTLVTVSS |
| 539C-R0010-F05 | 539C-M0020-F11 | EVQLLESGGGLVQPGGSLRLSCAASGFTFSWYAMVWVRQAPGKGLEWVSGISP |
| | | SGGLTSYADSVKGRFTISRDNSKNTLYLQMNSLRAEDTAVYYCARKQMSPDYY |
| | | YGMDVWGQGTTVTVSS |
| 539C-R0010-F06 | 539C-M0016-F12 | EVQLLESGGGLVQPGGSLRLSCAASGFTFSDYHMSWVRQAPGKGLEWVSGISS |
| | | SGGETFYADSVKGRFTISRDNSKNTLYLQMNSLRAEDTAVYYCASGSHRDGYN |
| | | LGYFDYWGQGTLVTVSS |
| 539C-R0004-G04 | 539C-M0008-D09 | EVQLLESGGGLVQPGGSLRLSCAASGFTFSDYDMHWVRQAPGKGLEWVSGISP |
| | | SGGWTWYADSVKGRFTISRDNSKNTLYLQMNSLRAEDTAVYYCARDWYSSGLD |
| | | YWGQGTLVTVSS |

### REFERENCES

The contents of all cited references including literature references, issued patents, published or non-published patent applications cited throughout this application as well as those listed below are hereby expressly incorporated by reference in their entireties. In case of conflict, the present application, including any definitions herein, will control.

Tometta M., et. al. Isolation of human anti-idiotypic antibodies by phage display for clinical immune response assays. J Immunol Methods. 2007; 328(1-2):34-44.

Goletz S., et. al. Selection of large diversities of anti-idiotypic antibody fragments by phage display. J Mol. Biol. 2002; 315(5):1087-97.

Macias A., et. al. Novel cross-reactive anti-idiotype antibodies with properties close to the human intravenous immunoglobulin (IVIg). Hybridoma. 1999 June; 18(3):263-72.

de Cerio A L, et al., Anti-idiotype antibodies in cancer treatment. Oncogene. 2007; 26(25):3594-602. Review.

### EQUIVALENTS

A number of embodiments of the invention have been described. Nevertheless, it will be understood that various modifications may be made without departing from the spirit and scope of the invention. Accordingly, other embodiments are within the scope of the following claims.

1. An isolated protein comprising a heavy chain (HC) immunoglobulin variable domain sequence and a light chain (LC) immunoglobulin variable domain sequence, wherein the HC and LC immunoglobulin variable domain sequences form an antigen binding site that binds to an anti-MMP-14 antibody; and the protein comprises one or more of the following characteristics:
(a) a human CDR or human framework region;
(b) the HC immunoglobulin variable domain sequence comprises one or more CDRs that are at least 85% identical to a CDR of a HC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04;
(c) the LC immunoglobulin variable domain sequence comprises one or more CDRs that are at least 85% identical to a CDR of a LC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04;
(d) the LC immunoglobulin variable domain sequence is at least 85% identical to a LC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04;
(e) the HC immunoglobulin variable domain sequence is at least 85% identical to a HC variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04; and
(f) the protein binds an epitope that overlaps with an epitope bound by 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04.

2. The protein of claim 1, wherein the anti-MMP-14 antibody is DX-2400.

3. An isolated nucleic acid comprising a sequence that encodes a polypeptide that comprises a sequence at least 80% identical to the sequence of a variable domain of 539C-M0016-E11; 539C-M0021-E01; 539C-R0010-A01; 539C-R0010-B01; 539C-R0010-B06; 539C-R0010-C05; 539C-R0010-C06; 539C-R0010-D06; 539C-R0010-E06; 539C-R0010-F05; 539C-R0010-F06; or 539C-R0004-G04.

4. A vector comprising the nucleic acid sequence of claim 3.

5. A host cell comprising the nucleic acid of claim 3.

6. An isolated nucleic acid comprising a sequence that encodes a polypeptide comprising the HC or the LC immunoglobulin variable domain of the protein of claim 1.

7. A vector comprising the nucleic acid sequence of claim 6.

8. A host cell comprising the nucleic acid of claim 6.

9. A method of detecting an anti-MMP-14 antibody in a biological sample, the method comprising: contacting the sample with the protein of claim 1; and detecting an interaction between the protein and the anti-MMP-14 antibody if present.

10. The method of claim 9, wherein the anti-MMP-14 antibody is DX-2400.

11. A method of detecting an anti-MMP-14 antibody in a subject, the method comprising:
administering the protein of claim 1, that further comprises a detectable label, to a subject; and
detecting the label in the subject.

12. The method of claim 11, wherein the anti-MMP-14 antibody is DX-2400.

13. A method of treating or preventing therapeutic antibody poisoning, the method comprising: administering the protein of claim 1 to a subject having poisoning or at risk of developing poisoning.

14. The method of claim 13, wherein the therapeutic antibody is DX-2400.

15. A method of purifying or removing an anti-MMP-14 antibody from a solution (e.g., a cell extract or biological sample), the method comprising:
contacting the solution with a protein of claim 1; and
eluting the anti-MMP-14 antibody that binds to the protein of claim 1.

16. The method of claim 15, wherein the anti-MMP-14 antibody is DX-2400.


SEQUENCE LISTING (232 sequences)

SEQ ID NO: 1 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 2 - protein, 582 aa, Mus musculus
MSPAPRPSRSLLLPLLTLGTALASLGWAQGSNFSPEAWLQQYGYLPPGDLRTHTQRSPQSLSAAIAAMQKFYGLQVTGKADLATMMAMRRPRCGVPDKFGTEIKANVRRKRYAIQGLKWQHNEITFCIQNYTPKVGEYATFEAIRKAFRVWESATPLRFREVPYAYIREGHEKQADIMILFAEGFHGDSTPFDGEGGFLAHAYFPGPNIGGDTHFDSAEPWTVQNEDLNGNDIFLVAVHELGHALGLEHSNDPSAIMSPFYQWMDTENFVLPDDDRRGIQQLYGSKSGSPTKMPPQPRTTSRPSVPDKPKNPAYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEEFRAVDSEYPKNIKVWEGIPESPRGSFMGSDEVFTYFYKGNKYWKFNNQKLKVEPGYPKSALRDWMGCPSGRRPDEGTEEETEVIIIEVDEEGSGAVSAAAVVLPVLLLLLVLAVGLAVFFFRRHGTPKRLLYCQRSLLDKV

SEQ ID NO: 3 - protein, 234 aa, Artificial Sequence
  Description of Artificial Sequence: Synthetic polypeptide
MKKLLFAIPLVVPFYSHSAQDIQMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 248 aa, Artificial Sequence
  Description of Artificial Sequence: Synthetic polypeptide
MKKLLFAIPLVVPFVAQPAMAEVQLLESGGGLVQPGGSLRLSCAASGFTFSFYVMDWVRQAPGKGLEWVSGIGPSGGSTDYADSVKGRFTISRDNSKNTLYLQMNSLRAEDTAVYYCTRIRYDSSGYPTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - protein, 234 aa, Artificial Sequence
  Description of Artificial Sequence: Synthetic polypeptide
MKKLLFAIPLVVPFYSHSAQDIQMTQSPSSLSASEGDRITLTCRASQSISIYLNWYQQKPGRAPKLLMYAATTLQSGVPSRFSGSGSGTDFTLTISGLRPEDFATYYCQQSYDIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 244 aa, Artificial Sequence
  Description of Artificial Sequence: Synthetic polypeptide
MKKLLFAIPLVVPFVAQPAMAEVQLLESGGGLVQPGGSLRLSCAASGFTFSVYPMIWVRQAPGKGLEWVSYISSSGGQTWYADSVKGRFTISRDNSKNTLYLQMNSLRAEDTATYYCARGDDYDSSGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 7 - protein, 115 aa, Artificial Sequence
  Description of Artificial Sequence: Synthetic polypeptide
EVQLLESGGGLVQPGGSLRLSCAASGFTFSLYSMNWVRQAPGKGLEWVSSIYSSGGSTLYADSVKGRFTISRDNSKNTLYLQMNSLRAEDTAVYYCARGRAFDIWGQGTMVTVSS

SEQ ID NO: 8 - protein, 108 aa, Artificial Sequence
  Description of Artificial Sequence: Synthetic polypeptide
DIQMTQSPSSLSASVGDRVTITCRASQSVGTYLNWYQQKPGKAPKLLIYATSNLRSGVPSRFSGSGSGTDFTLTISSLQPEDFATYYCQQSYSIPRFTFGPGTKVDIK

SEQ ID NO: 9 - protein, 6 aa, Artificial Sequence
  Description of Artificial Sequence: Synthetic peptide
FYSHSA

SEQ ID NO: 10 - protein, 6 aa, Artificial Sequence
  Description of Artificial Sequence: Synthetic peptide
FYSHSA

SEQ ID NO: 11 - protein, 6 aa, Artificial Sequence
  Description of Artificial Sequence: Synthetic peptide
FYSHSA

SEQ ID NO: 12 - protein, 6 aa, Artificial Sequence
  Description of Artificial Sequence: Synthetic peptide
FYSHSA

SEQ ID NO: 13 - protein, 6 aa, Artificial Sequence
  Description of Artificial Sequence: Synthetic peptide
FYSHSA

SEQ ID NO: 14 - protein, 6 aa, Artificial Sequence
  Description of Artificial Sequence: Synthetic peptide
FYSHSA

SEQ ID NO: 15 - protein, 6 aa, Artificial Sequence
  Description of Artificial Sequence: Synthetic peptide
FYSHSA

SEQ ID NO: 16 - protein, 6 aa, Artificial Sequence
  Description of Artificial Sequence: Synthetic peptide
FYSHSA

SEQ ID NO: 17 - protein, 6 aa, Artificial Sequence
  Description of Artificial Sequence: Synthetic peptide
FYSHSA

SEQ ID NO: 18 - protein, 6 aa, Artificial Sequence
  Description of Artificial Sequence: Synthetic peptide
FYSHSA

SEQ ID NO: 19 - protein, 24 aa, Artificial Sequence
  Description of Artificial Sequence: Synthetic peptide
QDIQMTQSPATLSLSPGERATLSC

SEQ ID NO: 20 - protein, 24 aa, Artificial Sequence
  Description of Artificial Sequence: Synthetic peptide
QDIQMTQSPSSLSASEGDRITLTC

SEQ ID NO: 21 - protein, 24 aa, Artificial Sequence
  Description of Artificial Sequence: Synthetic peptide
QDIQMTQSPATLSVSPGENATLSC

SEQ ID NO: 22 - protein, 24 aa, Artificial Sequence
  Description of Artificial Sequence: Synthetic peptide
QDIQMTQSPSSLSASVGDRVTITC

SEQ ID NO: 23 - protein, 24 aa, Artificial Sequence
  Description of Artificial Sequence: Synthetic peptide
QDIQMTQSPSSLSASVGDRVTITC

SEQ ID NO: 24 - protein, 22 aa, Artificial Sequence
  Description of Artificial Sequence: Synthetic peptide
QYELTQPHSVSESPGKTVTISC

SEQ ID NO: 25 - protein, 22 aa, Artificial Sequence
  Description of Artificial Sequence: Synthetic peptide
QSALTQPASVSGSPGQSITISC

SEQ ID NO: 26 - protein, 22 aa, Artificial Sequence
  Description of Artificial Sequence: Synthetic peptide
QSVLTQPPSVSVAPGQTATISC

SEQ ID NO: 27 - protein, 24 aa, Artificial Sequence
  Description of Artificial Sequence: Synthetic peptide
QDIQMTQSPASLSASVGDRVTITC

SEQ ID NO: 28 - protein, 22 aa, Artificial Sequence
  Description of Artificial Sequence: Synthetic peptide
QYELTQPPSVSVAPGQTARIIC

SEQ ID NO: 29 - protein, 11 aa, Artificial Sequence
  Description of Artificial Sequence: Synthetic peptide
RASQSVSSYLA

SEQ ID NO: 30 - protein, 11 aa, Artificial Sequence
  Description of Artificial Sequence: Synthetic peptide
RASQSISIYLN

SEQ ID NO: 31 - protein, 11 aa, Artificial Sequence
  Description of Artificial Sequence: Synthetic peptide
RASQSVASNLA

SEQ ID NO: 32 - protein, 11 aa, Artificial Sequence
  Description of Artificial Sequence: Synthetic peptide
LPSQDISVYLN

SEQ ID NO: 33 - protein, 11 aa, Artificial Sequence
  Description of Artificial Sequence: Synthetic peptide
RASQNINTFLN

SEQ ID NO: 34 - protein, 13 aa, Artificial Sequence
  Description of Artificial Sequence: Synthetic peptide
TRSNGTIDNSFVQ

SEQ ID NO: 35 - protein, 14 aa, Artificial Sequence
  Description of Artificial Sequence: Synthetic peptide
TGTSSDVGGYKLVS

SEQ ID NO: 36 - protein, 11 aa, Artificial Sequence
  Description of Artificial Sequence: Synthetic peptide
EGDNIGSKSVH

SEQ ID NO: 37 - protein, 11 aa, Artificial Sequence
  Description of Artificial Sequence: Synthetic peptide
QASRDIGEYLN

SEQ ID NO: 38 - protein, 11 aa, Artificial Sequence
  Description of Artificial Sequence: Synthetic peptide
GGNDIGSKGVH

SEQ ID NO: 39 - protein, 15 aa, Artificial Sequence
  Description of Artificial Sequence: Synthetic peptide
WYQQKPGQAPRLLIY

SEQ ID NO: 40 - protein, 15 aa, Artificial Sequence
  Description of Artificial Sequence: Synthetic peptide
WYQQKPGRAPKLLMY

SEQ ID NO: 41 - protein, 15 aa, Artificial Sequence
  Description of Artificial Sequence: Synthetic peptide
WYQQKPGQAPRLLIY

SEQ ID NO: 42 - protein, 15 aa, Artificial Sequence
  Description of Artificial Sequence: Synthetic peptide
WYQQKPGEAPKLLIS

SEQ ID NO: 43 - protein, 15 aa, Artificial Sequence
  Description of Artificial Sequence: Synthetic peptide
WYQQKPGKAPKLLIY

SEQ ID NO: 44 - protein, 15 aa, Artificial Sequence
  Description of Artificial Sequence: Synthetic peptide
WYQQRPGSAPTTVIY

SEQ ID NO: 45 - protein, 15 aa, Artificial Sequence
  Description of Artificial Sequence: Synthetic peptide
WYQQHPGKVPKLIIS

SEQ ID NO: 46 - protein, 15 aa, Artificial Sequence
  Description of Artificial Sequence: Synthetic peptide
WYQQRPGQAPVLVVY

SEQ ID NO: 47 - protein, 15 aa, Artificial Sequence
  Description of Artificial Sequence: Synthetic peptide
WYQQKAGKPPKLLIS

SEQ ID NO: 48 - protein, 15 aa, Artificial Sequence
  Description of Artificial Sequence: Synthetic peptide
WYHQKAGQAPVLLVY

SEQ ID NO: 49 - protein, 7 aa, Artificial Sequence
  Description of Artificial Sequence: Synthetic peptide
DASNRAT

SEQ ID NO: 50 - protein, 7 aa, Artificial Sequence
  Description of Artificial Sequence: Synthetic peptide
AATTLQS

SEQ ID NO: 51 - protein, 7 aa, Artificial Sequence
  Description of Artificial Sequence: Synthetic peptide
GASTRAT

SEQ ID NO: 52 - protein, 7 aa, Artificial Sequence
  Description of Artificial Sequence: Synthetic peptide
ATSDLQS

SEQ ID NO: 53 - protein, 7 aa, Artificial Sequence
  Description of Artificial Sequence: Synthetic peptide
GASSLQS

SEQ ID NO: 54 - protein, 7 aa, Artificial Sequence
  Description of Artificial Sequence: Synthetic peptide
EDTQRPS

SEQ ID NO: 55 - protein, 7 aa, Artificial Sequence
  Description of Artificial Sequence: Synthetic peptide
EVSYRPS

SEQ ID NO: 56 - protein, 7 aa, Artificial Sequence
  Description of Artificial Sequence: Synthetic peptide
DDYDRPS

SEQ ID NO: 57 - protein, 7 aa, Artificial Sequence
  Description of Artificial Sequence: Synthetic peptide
DATTLET

SEQ ID NO: 58 - protein, 7 aa, Artificial Sequence
  Description of Artificial Sequence: Synthetic peptide
DNTDRPS

SEQ ID NO: 59 - protein, 32 aa, Artificial Sequence
  Description of Artificial Sequence: Synthetic polypeptide
GIPARFSGSGSGTDFTLTISSLEPEDFAVYYC

SEQ ID NO: 60 - protein, 32 aa, Artificial Sequence
  Description of Artificial Sequence: Synthetic polypeptide
GVPSRFSGSGSGTDFTLTISGLRPEDFATYYC

SEQ ID NO: 61 - protein, 32 aa, Artificial Sequence
  Description of Artificial Sequence: Synthetic polypeptide
GAPARFSGSGSETDFTLTISSLQSEDFAVYYC

SEQ ID NO: 62 - protein, 32 aa, Artificial Sequence
  Description of Artificial Sequence: Synthetic polypeptide
GVPSRFGGSGYGTDFSLTITSLQREDFATYYC

SEQ ID NO: 63 - protein, 32 aa, Artificial Sequence
  Description of Artificial Sequence: Synthetic polypeptide
GVPSRFSGSGSGTDFTLTISSLQPEDFATYYC

SEQ ID NO: 64 - protein, 34 aa, Artificial Sequence
  Description of Artificial Sequence: Synthetic polypeptide
GVPDRFSGSIDSSSNSASLTISGLKTEDEADYYC

SEQ ID NO: 65 - protein, 32 aa, Artificial Sequence
  Description of Artificial Sequence: Synthetic polypeptide
GVSNRFSGSKSGNAASLTISGLQAEDEADYYC

SEQ ID NO: 66 - protein, 32 aa, Artificial Sequence
  Description of Artificial Sequence: Synthetic polypeptide
GIPERFSGFNSGNTATLTIYRVEAGDEADFYC

SEQ ID NO: 67 - protein, 32 aa, Artificial Sequence
  Description of Artificial Sequence: Synthetic polypeptide
GVPSRFSGTGSGTEFLFTISSVEPEDFATYYC

SEQ ID NO: 68 - protein, 32 aa, Artificial Sequence
  Description of Artificial Sequence: Synthetic polypeptide
GIPERFSGSNSGNTAALTITRVEAGDEADYFC

SEQ ID NO: 69 - protein, 9 aa, Artificial Sequence
  Description of Artificial Sequence: Synthetic peptide
QQRSNWPLT

SEQ ID NO: 70 - protein, 9 aa, Artificial Sequence
  Description of Artificial Sequence: Synthetic peptide
QQSYDIPLT

SEQ ID NO: 71 - protein, 10 aa, Artificial Sequence
  Description of Artificial Sequence: Synthetic peptide
QQYHNWPPWT

SEQ ID NO: 72 - protein, 9 aa, Artificial Sequence
  Description of Artificial Sequence: Synthetic peptide
QQSYSLPFT

SEQ ID NO: 73 - protein, 10 aa, Artificial Sequence
  Description of Artificial Sequence: Synthetic peptide
QQSSDALVYN

SEQ ID NO: 74 - protein, 10 aa, Artificial Sequence
  Description of Artificial Sequence: Synthetic peptide
QSYDSTNRGV

SEQ ID NO: 75 - protein, 10 aa, Artificial Sequence
  Description of Artificial Sequence: Synthetic peptide
SSYTSTSTWV

SEQ ID NO: 76 - protein, 11 aa, Artificial Sequence
  Description of Artificial Sequence: Synthetic peptide
QVRDSRTEERV

SEQ ID NO: 77 - protein, 10 aa, Artificial Sequence
  Description of Artificial Sequence: Synthetic peptide
QQYDDLTWIS

SEQ ID NO: 78 - protein, 11 aa, Artificial Sequence
  Description of Artificial Sequence: Synthetic peptide
QVWDSTGEHAV

SEQ ID NO: 79 - protein, 10 aa, Artificial Sequence
  Description of Artificial Sequence: Synthetic peptide
FGGGTKVEIK

SEQ ID NO: 80 - protein, 10 aa, Artificial Sequence
  Description of Artificial Sequence: Synthetic peptide
FGGGTKVEIK

SEQ ID NO: 81 - protein, 10 aa, Artificial Sequence
  Description of Artificial Sequence: Synthetic peptide
FGQGTKVEIK

SEQ ID NO: 82 - protein, 10 aa, Artificial Sequence
  Description of Artificial Sequence: Synthetic peptide
FGGGTRVEIK

SEQ ID NO: 83 - protein, 10 aa, Artificial Sequence
  Description of Artificial Sequence: Synthetic peptide
FGQGTKLEIK

SEQ ID NO: 84 - protein, 10 aa, Artificial Sequence
  Description of Artificial Sequence: Synthetic peptide
FGGGTKLTVL

SEQ ID NO: 85 - protein, 10 aa, Artificial Sequence
  Description of Artificial Sequence: Synthetic peptide
FGGGTKLTVL

SEQ ID NO: 86 - protein, 10 aa, Artificial Sequence
  Description of Artificial Sequence: Synthetic peptide
FGGGTKLTVL

SEQ ID NO: 87 - protein, 10 aa, Artificial Sequence
  Description of Artificial Sequence: Synthetic peptide
FGPGTRLDVK

SEQ ID NO: 88 - protein, 10 aa, Artificial Sequence
  Description of Artificial Sequence: Synthetic peptide
FGGGTKLTVL

SEQ ID NO: 89 - protein, 7 aa, Artificial Sequence
  Description of Artificial Sequence: Synthetic peptide
RTVAAPS

SEQ ID NO: 90 - protein, 7 aa, Artificial Sequence
  Description of Artificial Sequence: Synthetic peptide
RTVAAPS

SEQ ID NO: 91 - protein, 7 aa, Artificial Sequence
  Description of Artificial Sequence: Synthetic peptide
RTVAAPS

SEQ ID NO: 92 - protein, 7 aa, Artificial Sequence
  Description of Artificial Sequence: Synthetic peptide
RTVAAPS

SEQ ID NO: 93 - protein, 7 aa, Artificial Sequence
  Description of Artificial Sequence: Synthetic peptide
RTVAAPS

SEQ ID NO: 94 - protein, 7 aa, Artificial Sequence
  Description of Artificial Sequence: Synthetic peptide
GQPKAAP

SEQ ID NO: 95 - protein, 7 aa, Artificial Sequence
  Description of Artificial Sequence: Synthetic peptide
GQPKAAP

SEQ ID NO: 96 - protein, 7 aa, Artificial Sequence
  Description of Artificial Sequence: Synthetic peptide
GQPKAAP

SEQ ID NO: 97 - protein, 7 aa, Artificial Sequence
  Description of Artificial Sequence: Synthetic peptide
RTVAAPS

SEQ ID NO: 98 - protein, 7 aa, Artificial Sequence
  Description of Artificial Sequence: Synthetic peptide
GQPKAAP

SEQ ID NO: 99 - protein, 21 aa, Artificial Sequence
  Description of Artificial Sequence: Synthetic peptide
MKKLLFAIPLVVPFVAQPAMA

SEQ ID NO: 100 - protein, 21 aa, Artificial Sequence
  Description of Artificial Sequence: Synthetic peptide
MKKLLFAIPLVVPFVAQPASA

SEQ ID NO: 101 - protein, 21 aa, Artificial Sequence
  Description of Artificial Sequence: Synthetic peptide
MKKLLFAIPLVVPFVAQPASA

SEQ ID NO: 102 - protein, 21 aa, Artificial Sequence
  Description of Artificial Sequence: Synthetic peptide
MKKLLFAIPLVVPFVAQPASA

SEQ ID NO: 103 - protein, 21 aa, Artificial Sequence
  Description of Artificial Sequence: Synthetic peptide
MKKLLFAIPLVVPFVAQPASA

SEQ ID NO: 104 - protein, 21 aa, Artificial Sequence
  Description of Artificial Sequence: Synthetic peptide
MKKLLFAIPLVVPFVAQPASA

SEQ ID NO: 105 - protein, 21 aa, Artificial Sequence
  Description of Artificial Sequence: Synthetic peptide
MKKLLFAIPLVVPFVAQPASA

SEQ ID NO: 106 - protein, 21 aa, Artificial Sequence
  Description of Artificial Sequence: Synthetic peptide
MKKLLFAIPLVVPFVAQPASA

SEQ ID NO: 107 - protein, 21 aa, Artificial Sequence
  Description of Artificial Sequence: Synthetic peptide
MKKLLFAIPLVVPFVAQPAMA

SEQ ID NO: 108 - protein, 21 aa, Artificial Sequence
  Description of Artificial Sequence: Synthetic peptide
MKKLLFAIPLVVPFVAQPAMA

SEQ ID NO: 109 - protein, 30 aa, Artificial Sequence
  Description of Artificial Sequence: Synthetic polypeptide
EVQLLESGGGLVQPGGSLRLSCAASGFTFS

SEQ ID NO: 110 - protein, 30 aa, Artificial Sequence; identical to SEQ ID NO: 109
  Description of Artificial Sequence: Synthetic polypeptide

SEQ ID NO: 111 - protein, 30 aa, Artificial Sequence; identical to SEQ ID NO: 109
  Description of Artificial Sequence: Synthetic polypeptide

SEQ ID NO: 112 - protein, 30 aa, Artificial Sequence; identical to SEQ ID NO: 109
  Description of Artificial Sequence: Synthetic polypeptide

SEQ ID NO: 113 - protein, 30 aa, Artificial Sequence; identical to SEQ ID NO: 109
  Description of Artificial Sequence: Synthetic polypeptide

SEQ ID NO: 114 - protein, 30 aa, Artificial Sequence; identical to SEQ ID NO: 109
  Description of Artificial Sequence: Synthetic polypeptide

SEQ ID NO: 115 - protein, 30 aa, Artificial Sequence; identical to SEQ ID NO: 109
  Description of Artificial Sequence: Synthetic polypeptide

SEQ ID NO: 116 - protein, 30 aa, Artificial Sequence; identical to SEQ ID NO: 109
  Description of Artificial Sequence: Synthetic polypeptide

SEQ ID NO: 117 - protein, 30 aa, Artificial Sequence; identical to SEQ ID NO: 109
  Description of Artificial Sequence: Synthetic polypeptide

SEQ ID NO: 118 - protein, 30 aa, Artificial Sequence; identical to SEQ ID NO: 109
  Description of Artificial Sequence: Synthetic polypeptide

SEQ ID NO: 119 - protein, 5 aa, Artificial Sequence
  Description of Artificial Sequence: Synthetic peptide
FYVMD

SEQ ID NO: 120 - protein, 5 aa, Artificial Sequence
  Description of Artificial Sequence: Synthetic peptide
VYPMI

SEQ ID NO: 121 - protein, 5 aa, Artificial Sequence
  Description of Artificial Sequence: Synthetic peptide
EYQMN

SEQ ID NO: 122 - protein, 5 aa, Artificial Sequence
  Description of Artificial Sequence: Synthetic peptide
GYGMG

SEQ ID NO: 123 - protein, 5 aa, Artificial Sequence
  Description of Artificial Sequence: Synthetic peptide
SYNMA

SEQ ID NO: 124 - protein, 5 aa, Artificial Sequence
  Description of Artificial Sequence: Synthetic peptide
DYSMY

SEQ ID NO: 125 - protein, 5 aa, Artificial Sequence
  Description of Artificial Sequence: Synthetic peptide
DYWMF

SEQ ID NO: 126 - protein, 5 aa, Artificial Sequence
  Description of Artificial Sequence: Synthetic peptide
WYAMV

SEQ ID NO: 127 - protein, 5 aa, Artificial Sequence
  Description of Artificial Sequence: Synthetic peptide
DYHMS

SEQ ID NO: 128 - protein, 5 aa, Artificial Sequence
  Description of Artificial Sequence: Synthetic peptide
DYDMH

SEQ ID NO: 129 - protein, 14 aa, Artificial Sequence
  Description of Artificial Sequence: Synthetic peptide
WVRQAPGKGLEWVS

SEQ ID NO: 130 - protein, 14 aa, Artificial Sequence
  Description of Artificial Sequence: Synthetic peptide
WVRQAPGKGLEWVS

SEQ ID NO: 131 - protein, 14 aa, Artificial Sequence
  Description of Artificial Sequence: Synthetic peptide
WVRQAPGKGLEWVS

SEQ ID NO: 132 - protein, 14 aa, Artificial Sequence
  Description of Artificial Sequence: Synthetic peptide
WVRQAPGKGLEWVS

SEQ ID NO: 133 - protein, 14 aa, Artificial Sequence
  Description of Artificial Sequence: Synthetic peptide
WVRQAPGKGLEWVS

SEQ ID NO: 134 - protein, 14 aa, Artificial Sequence
  Description of Artificial Sequence: Synthetic peptide
WVRQAPGKGLEWVS

SEQ ID NO: 135 - protein, 14 aa, Artificial Sequence
  Description of Artificial Sequence: Synthetic peptide
WVRQAPGKGLEWVS

SEQ ID NO: 136 - protein, 14 aa, Artificial Sequence
  Description of Artificial Sequence: Synthetic peptide
WVRQAPGKGLEWVS

SEQ ID NO: 137 - protein, 14 aa, Artificial Sequence
  Description of Artificial Sequence: Synthetic peptide
WVRQAPGKGLEWVS

SEQ ID NO: 138 - protein, 14 aa, Artificial Sequence
  Description of Artificial Sequence: Synthetic peptide
WVRQAPGKGLEWVS

SEQ ID NO: 139 - protein, 17 aa, Artificial Sequence
  Description of Artificial Sequence: Synthetic peptide
GIGPSGGSTDYADSVKG

SEQ ID NO: 140 - protein, 17 aa, Artificial Sequence
  Description of Artificial Sequence: Synthetic peptide
YISSSGGQTWYADSVKG

SEQ ID NO: 141 - protein, 17 aa, Artificial Sequence
  Description of Artificial Sequence: Synthetic peptide
SIYSSGGGTAYADSVKG

SEQ ID NO: 142 - protein, 17 aa, Artificial Sequence
  Description of Artificial Sequence: Synthetic peptide
YIVPSGGATDYADSVKG

SEQ ID NO: 143 - protein, 17 aa, Artificial Sequence
  Description of Artificial Sequence: Synthetic peptide
VIVPSGGLTGYADSVKG

SEQ ID NO: 144 - protein, 17 aa, Artificial Sequence
  Description of Artificial Sequence: Synthetic peptide
YISSSGGNTEYADSVKG

SEQ ID NO: 145 - protein, 17 aa, Artificial Sequence
  Description of Artificial Sequence: Synthetic peptide
GIGPSGGIISYADSVKG

SEQ ID NO: 146 - protein, 17 aa, Artificial Sequence
  Description of Artificial Sequence: Synthetic peptide
GISPSGGLTSYADSVKG

SEQ ID NO: 147 - protein, 17 aa, Artificial Sequence
  Description of Artificial Sequence: Synthetic peptide
GISSSGGETFYADSVKG

SEQ ID NO: 148 - protein, 17 aa, Artificial Sequence
  Description of Artificial Sequence: Synthetic peptide
GISPSGGWTWYADSVKG

SEQ ID NO: 149 - protein, 32 aa, Artificial Sequence
  Description of Artificial Sequence: Synthetic polypeptide
RFTISRDNSKNTLYLQMNSLRAEDTAVYYCTR

SEQ ID NO: 150 - protein, 32 aa, Artificial Sequence
  Description of Artificial Sequence: Synthetic polypeptide
RFTISRDNSKNTLYLQMNSLRAEDTATYYCAR

SEQ ID NO: 151 - protein, 32 aa, Artificial Sequence
  Description of Artificial Sequence: Synthetic polypeptide
RFTISRDNSKNTLYLQMNSLRAEDTAVYYCAK

SEQ ID NO: 152 - protein, 32 aa, Artificial Sequence
  Description of Artificial Sequence: Synthetic polypeptide
RFTISRDNSKNTLYLQMNSLRAEDTAVYYCAR

SEQ ID NO: 153 - protein, 32 aa, Artificial Sequence; identical to SEQ ID NO: 152
  Description of Artificial Sequence: Synthetic polypeptide

SEQ ID NO: 154 - protein, 32 aa, Artificial Sequence
  Description of Artificial Sequence: Synthetic polypeptide
RFTISRDNSKNTLYLQMNSLRAEDTAMYYCAT

SEQ ID NO: 155 - protein, 32 aa, Artificial Sequence; identical to SEQ ID NO: 151
  Description of Artificial Sequence: Synthetic polypeptide

SEQ ID NO: 156 - protein, 32 aa, Artificial Sequence; identical to SEQ ID NO: 152
  Description of Artificial Sequence: Synthetic polypeptide

SEQ ID NO: 157 - protein, 32 aa, Artificial Sequence
  Description of Artificial Sequence: Synthetic polypeptide
RFTISRDNSKNTLYLQMNSLRAEDTAVYYCAS

SEQ ID NO: 158 - protein, 32 aa, Artificial Sequence; identical to SEQ ID NO: 152
  Description of Artificial Sequence: Synthetic polypeptide

SEQ ID NO: 159 - protein, 15 aa, Artificial Sequence
  Description of Artificial Sequence: Synthetic peptide
IRYDSSGYPTDAFDI

SEQ ID NO: 160 - protein, 11 aa, Artificial Sequence
  Description of Artificial Sequence: Synthetic peptide
GDDYDSSGPDY

SEQ ID NO: 161 - protein, 14 aa, Artificial Sequence
  Description of Artificial Sequence: Synthetic peptide
DQGDGYNYGVGLDY

SEQ ID NO: 162 - protein, 11 aa, Artificial Sequence
  Description of Artificial Sequence: Synthetic peptide
AYYYDSSGFDI

SEQ ID NO: 163 - protein, 17 aa, Artificial Sequence
  Description of Artificial Sequence: Synthetic peptide
ERGGFYSSGWYRYWFDP

SEQ ID NO: 164 - protein, 15 aa, Artificial Sequence
  Description of Artificial Sequence: Synthetic peptide
TPSIWLGDLLENGPY

SEQ ID NO: 165 - protein, 10 aa, Artificial Sequence
  Description of Artificial Sequence: Synthetic peptide
YGDYVSGFDY

SEQ ID NO: 166 - protein, 13 aa, Artificial Sequence
  Description of Artificial Sequence: Synthetic peptide
KQMSPDYYYGMDV

SEQ ID NO: 167 - protein, 14 aa, Artificial Sequence
  Description of Artificial Sequence: Synthetic peptide
GSHRDGYNLGYFDY

SEQ ID NO: 168 - protein, 9 aa, Artificial Sequence
  Description of Artificial Sequence: Synthetic peptide
DWYSSGLDY

SEQ ID NO: 169 - protein, 11 aa, Artificial Sequence
  Description of Artificial Sequence: Synthetic peptide
WGQGTMVTVSS

SEQ ID NO: 170 - protein, 11 aa, Artificial Sequence
  Description of Artificial Sequence: Synthetic peptide
WGQGTLVTVSS

SEQ ID NO: 171 - protein, 11 aa, Artificial Sequence
  Description of Artificial Sequence: Synthetic peptide
WGQGTLVTVSS

SEQ ID NO: 172 - protein, 11 aa, Artificial Sequence
  Description of Artificial Sequence: Synthetic peptide
WGQGTMVTVSS

SEQ ID NO: 173 - protein, 11 aa, Artificial Sequence
  Description of Artificial Sequence: Synthetic peptide
WGQGTLVTVSS

SEQ ID NO: 174 - protein, 11 aa, Artificial Sequence
  Description of Artificial Sequence: Synthetic peptide
WGQGTLVTVSS

SEQ ID NO: 175 - protein, 11 aa, Artificial Sequence
  Description of Artificial Sequence: Synthetic peptide
WGQGTLVTVSS

SEQ ID NO: 176 - protein, 11 aa, Artificial Sequence
  Description of Artificial Sequence: Synthetic peptide
WGQGTTVTVSS

SEQ ID NO: 177 - protein, 11 aa, Artificial Sequence
  Description of Artificial Sequence: Synthetic peptide
WGQGTLVTVSS

SEQ ID NO: 178 - protein, 11 aa, Artificial Sequence
  Description of Artificial Sequence: Synthetic peptide
WGQGTLVTVSS

SEQ ID NO: 179 - protein, 17 aa, Artificial Sequence
  Description of Artificial Sequence: Synthetic peptide
ASTKGPSVFPLAPSSKS

SEQ ID NO: 180 - protein, 17 aa, Artificial Sequence
  Description of Artificial Sequence: Synthetic peptide
ASTKGPSVFPLAPSSKS

SEQ ID NO: 181 - protein, 17 aa, Artificial Sequence
  Description of Artificial Sequence: Synthetic peptide
ASTKGPSVFPLAPSSKS

SEQ ID NO: 182 - protein, 17 aa, Artificial Sequence
  Description of Artificial Sequence: Synthetic peptide
ASTKGPSVFPLAPSSKS

SEQ ID NO: 183 - protein, 17 aa, Artificial Sequence
  Description of Artificial Sequence: Synthetic peptide
ASTKGPSVFPLAPSSKS

SEQ ID NO: 184 - protein, 17 aa, Artificial Sequence
  Description of Artificial Sequence: Synthetic peptide
ASTKGPSVFPLAPSSKS

SEQ ID NO: 185 - protein, 17 aa, Artificial Sequence
  Description of Artificial Sequence: Synthetic peptide
ASTKGPSVFPLAPSSKS

SEQ ID NO: 186 - protein, 17 aa, Artificial Sequence
  Description of Artificial Sequence: Synthetic peptide
ASTKGPSVFPLAPSSKS

SEQ ID NO: 187 - protein, 17 aa, Artificial Sequence
  Description of Artificial Sequence: Synthetic peptide
ASTKGPSVFPLAPSSKS

SEQ ID NO: 188 - protein, 17 aa, Artificial Sequence
  Description of Artificial Sequence: Synthetic peptide
ASTKGPSVFPLAPSSKS

SEQ ID NO: 189 - protein, 108 aa, Artificial Sequence
  Description of Artificial Sequence: Synthetic polypeptide
QDIQMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 190 - protein, 108 aa, Artificial Sequence
  Description of Artificial Sequence: Synthetic polypeptide
QDIQMTQSPSSLSASEGDRITLTCRASQSISIYLNWYQQKPGRAPKLLMYAATTLQSGVPSRFSGSGSGTDFTLTISGLRPEDFATYYCQQSYDIPLTFGGGTKVEIK

SEQ ID NO: 191 - protein, 109 aa, Artificial Sequence
  Description of Artificial Sequence: Synthetic polypeptide
QDIQMTQSPATLSVSPGENATLSCRASQSVASNLAWYQQKPGQAPRLLIYGASTRATGAPARFSGSGSETDFTLTISSLQSEDFAVYYCQQYHNWPPWTFGQGTKVEIK

SEQ ID NO: 192 - protein, 108 aa, Artificial Sequence
  Description of Artificial Sequence: Synthetic polypeptide
QDIQMTQSPSSLSASVGDRVTITCLPSQDISVYLNWYQQKPGEAPKLLISATSDLQSGVPSRFGGSGYGTDFSLTITSLQREDFATYYCQQSYSLPFTFGGGTRVEIK

SEQ ID NO: 193 - protein, 109 aa, Artificial Sequence
  Description of Artificial Sequence: Synthetic polypeptide
QDIQMTQSPSSLSASVGDRVTITCRASQNINTFLNWYQQKPGKAPKLLIYGASSLQSGVPSRFSGSGSGTDFTLTISSLQPEDFATYYCQQSSDALVYNFGQGTKLEIK

SEQ ID NO: 194 - protein, 111 aa, Artificial Sequence
  Description of Artificial Sequence: Synthetic polypeptide
QYELTQPHSVSESPGKTVTISCTRSNGTIDNSFVQWYQQRPGSAPTTVIYEDTQRPSGVPDRFSGSIDSSSNSASLTISGLKTEDEADYYCQSYDSTNRGVFGGGTKLTVL

SEQ ID NO: 195 - protein, 110 aa, Artificial Sequence
  Description of Artificial Sequence: Synthetic polypeptide
QSALTQPASVSGSPGQSITISCTGTSSDVGGYKLVSWYQQHPGKVPKLIISEVSYRPSGVSNRFSGSKSGNAASLTISGLQAEDEADYYCSSYTSTSTWVFGGGTKLTVL

SEQ ID NO: 196 - protein, 108 aa, Artificial Sequence
  Description of Artificial Sequence: Synthetic polypeptide
QSVLTQPPSVSVAPGQTATISCEGDNIGSKSVHWYQQRPGQAPVLVVYDDYDRPSGIPERFSGFNSGNTATLTIYRVEAGDEADFYCQVRDSRTEERVFGGGTKLTVL

SEQ ID NO: 197 - protein, 109 aa, Artificial Sequence
  Description of Artificial Sequence: Synthetic polypeptide
QDIQMTQSPASLSASVGDRVTITCQASRDIGEYLNWYQQKAGKPPKLLISDATTLETGVPSRFSGTGSGTEFLFTISSVEPEDFATYYCQQYDDLTWISFGPGTRLDVK

SEQ ID NO: 198 - protein, 108 aa, Artificial Sequence
  Description of Artificial Sequence: Synthetic polypeptide
QYELTQPPSVSVAPGQTARIICGGNDIGSKGVHWYHQKAGQAPVLLVYDNTDRPSGIPERFSGSNSGNTAALTITRVEAGDEADYFCQVWDSTGEHAVFGGGTKLTVL

SEQ ID NO: 199 - protein, 124 aa, Artificial Sequence
  Description of Artificial Sequence: Synthetic polypeptide
EVQLLESGGGLVQPGGSLRLSCAASGFTFSFYVMDWVRQAPGKGLEWVSGIGPSGGSTDYADSVKGRFTISRDNSKNTLYLQMNSLRAEDTAVYYCTRIRYDSSGYPTDAFDIWGQGTMVTVSS

SEQ ID NO: 200 - protein, 120 aa, Artificial Sequence
  Description of Artificial Sequence: Synthetic polypeptide
EVQLLESGGGLVQPGGSLRLSCAASGFTFSVYPMIWVRQAPGKGLEWVSYISSSGGQTWYADSVKGRFTISRDNSKNTLYLQMNSLRAEDTATYYCARGDDYDSSGPDYWGQGTLVTVSS

SEQ ID NO: 201 - protein, 123 aa, Artificial Sequence
  Description of Artificial Sequence: Synthetic polypeptide
EVQLLESGGGLVQPGGSLRLSCAASGFTFSEYQMNWVRQAPGKGLEWVSSIYSSGGGTAYADSVKGRFTISRDNSKNTLYLQMNSLRAEDTAVYYCAKDQGDGYNYGVGLDYWGQGTLVTVSS

SEQ ID NO: 202 - protein, 120 aa, Artificial Sequence
  Description of Artificial Sequence: Synthetic polypeptide
EVQLLESGGGLVQPGGSLRLSCAASGFTFSGYGMGWVRQAPGKGLEWVSYIVPSGGATDYADSVKGRFTISRDNSKNTLYLQMNSLRAEDTAVYYCARAYYYDSSGFDIWGQGTMVTVSS

SEQ ID NO: 203 - protein, 126 aa, Artificial Sequence
  Description of Artificial Sequence: Synthetic polypeptide
EVQLLESGGGLVQPGGSLRLSCAASGFTFSSYNMAWVRQAPGKGLEWVSVIVPSGGLTGYADSVKGRFTISRDNSKNTLYLQMNSLRAEDTAVYYCARERGGFYSSGWYRYWFDPWGQGTLVTVSS

SEQ ID NO: 204 - protein, 124 aa, Artificial Sequence
  Description of Artificial Sequence: Synthetic polypeptide
EVQLLESGGGLVQPGGSLRLSCAASGFTFSDYSMYWVRQAPGKGLEWVSYISSSGGNTEYADSVKGRFTISRDNSKNTLYLQMNSLRAEDTAMYYCATTPSIWLGDLLENGPYWGQGTLVTVSS

SEQ ID NO: 205 - protein, 119 aa, Artificial Sequence
  Description of Artificial Sequence: Synthetic polypeptide
EVQLLESGGGLVQPGGSLRLSCAASGFTFSDYWMFWVRQAPGKGLEWVSGIGPSGGIISYADSVKGRFTISRDNSKNTLYLQMNSLRAEDTAVYYCAKYGDYVSGFDYWGQGTLVTVSS

SEQ ID NO: 206 - protein, 122 aa, Artificial Sequence
  Description of Artificial Sequence: Synthetic polypeptide
EVQLLESGGGLVQPGGSLRLSCAASGFTFSWYAMVWVRQAPGKGLEWVSGISPSGGLTSYADSVKGRFTISRDNSKNTLYLQMNSLRAEDTAVYYCARKQMSPDYYYGMDVWGQGTTVTVSS

SEQ ID NO: 207 - protein, 123 aa, Artificial Sequence
  Description of Artificial Sequence: Synthetic polypeptide
EVQLLESGGGLVQPGGSLRLSCAASGFTFSDYHMSWVRQAPGKGLEWVSGISSSGGETFYADSVKGRFTISRDNSKNTLYLQMNSLRAEDTAVYYCASGSHRDGYNLGYFDYWGQGTLVTVSS

SEQ ID NO: 208 - protein, 118 aa, Artificial Sequence
  Description of Artificial Sequence: Synthetic polypeptide
EVQLLESGGGLVQPGGSLRLSCAASGFTFSDYDMHWVRQAPGKGLEWVSGISPSGGWTWYADSVKGRFTISRDNSKNTLYLQMNSLRAEDTAVYYCARDWYSSGLDYWGQGTLVTVSS

SEQ ID NO: 209 - protein, 126 aa, Artificial Sequence
  Description of Artificial Sequence: Synthetic polypeptide
EVQLLESGGGLVQPGGSLRLSCAASGFTFSPYPMGWVRQAPGKGLEWVSSIVSSGGLTLYADSVKGRFTISRDNSKNTLYLQMNSLRAEDTAVYYCARGGRLYDILTGQGAPFDYWGQGTLVTVSS

SEQ ID NO: 210 - protein, 113 aa, Artificial Sequence
  Description of Artificial Sequence: Synthetic polypeptide
QDIQMTQSPLSLPVTPGEPASISCRSSQSLLHSNGYYYLDWYLQKPGQSPQLLIYLGSYRASGVPDRFSGSGSGTDFTLKISSVEAEDVGVYYCMQALQTPLTFGGGTRVDIK

SEQ ID NO: 211 - protein, 125 aa, Artificial Sequence
  Description of Artificial Sequence: Synthetic polypeptide
EVQLLESGGGLVQPGGSLRLSCAASGFTFSTYEMHWVRQAPGKGLEWVSSIYSSGGWTGYADSVKGRFTISRDNSKNTLYLQMNSLRAEDTAVYYCARSQQYYDFSSRYYGMDVWGQGTTVTVSS

SEQ ID NO: 212 - protein, 111 aa, Artificial Sequence
  Description of Artificial Sequence: Synthetic polypeptide
QSELTQPPSVSGTPGQRVTISCSGTSANIGRNAVHWYQQLPGTAPKLLIHSNNRRPSGVPDRFSGSKSGTSASLAISGLQSEDEADYYCAAWENSLNAFYVFGTGTKVTVL

SEQ ID NO: 213 - protein, 120 aa, Artificial Sequence
  Description of Artificial Sequence: Synthetic polypeptide
EVQLLESGGGLVQPGGSLRLSCAASGFTFSVYGMVWVRQAPGKGLEWVSVISSSGGSTWYADSVKGRFTISRDNSKNTLYLQMNSLRAEDTALYYCARPFSRRYGVFDYWGQGTLVTVSS

SEQ ID NO: 214 - protein, 108 aa, Artificial Sequence
  Description of Artificial Sequence: Syntheticy polypeptide
QDIQMTQSPSSLSASVGDRVTITCRASQGIRNFLAWYQQKPGKVPKLLVFGASALQSGVPSRFSGSGSGTDFTLTISGLQPEDVATYYCQKYNGVPLTFGGGTKVEIK

SEQ ID NO: 215 - protein, 125 aa, Artificial Sequence
  Description of Artificial Sequence: Synthetic polypeptide
EVQLLESGGGLVQPGGSLRLSCAASGFTFSHYEMFWVRQAPGKGLEWVSSISPSGGQTHYADSVKGRFTISRDNSKNTLYLQMNSLRAEDTAVYYCATDRTYYDFWSGYGPLWYWGQGTLVTVSS

SEQ ID NO: 216 - protein, 113 aa, Artificial Sequence
  Description of Artificial Sequence: Synthetic polypeptide
QDIQMTQSPLSLPVTLGESASVSCRSSQSLLHENGHNYLDWYLQKPGQSPQLLIYLGSNRASGVPDRFSGSGSGTDFTLKISRVEAEDVGVYYCMQSLKTPPTFGPGTKVEIK

SEQ ID NO: 217 - protein, 125 aa, Artificial Sequence
  Description of Artificial Sequence: Synthetic polypeptide
EVQLLESGGGLVQPGGSLRLSCAASGFTFSMYMMIWVRQAPGKGLEWVSSIYPSGGNTMYADSVKGRFTISRDNSKNTLYLQMNSLRAEDTAVYYCATGVLRYFDWDAGSGMDVWGQGTTVTVSS

SEQ ID NO: 218 - protein, 108 aa, Artificial Sequence
  Description of Artificial Sequence: Synthetic polypeptide
QDIQMTQSPSSLSASVGDRVTITCRASQGIRNDLGWYQQKPGKAPKRLIYVASSLQSGVPSRFSGSGSGTEFTLTISSLQPEDFATYYCLQHNSYPWTFGQGTKVEIK

SEQ ID NO: 219 - protein, 118 aa, Artificial Sequence
  Description of Artificial Sequence: Synthetic polypeptide
EVQLLESGGGLVQPGGSLRLSCAASGFTFSPYVMHWVRQAPGKGLEWVSSISPSGGWTYYADSVKGRFTISRDNSKNTLYLQMNSLRAEDMAVYYCARGTGAYGMDVWGQGTTVTVSS

SEQ ID NO: 220 - protein, 109 aa, Artificial Sequence
  Description of Artificial Sequence: Synthetic polypeptide
QDIQMTQSPGTLSLSPGDRATLSCGASQLVVSNYIAWYQQKPGQAPRLLMYAGSIRATGIPDRFSGSGSGTDFTLTISRLEPEDFAIYYCQQRSNWPWTFGQGTKVEIK

SEQ ID NO: 221 - protein, 115 aa, Artificial Sequence; identical to SEQ ID NO: 7
  Description of Artificial Sequence: Synthetic polypeptide

SEQ ID NO: 222 - protein, 109 aa, Artificial Sequence
  Description of Artificial Sequence: Synthetic polypeptide
QDIQMTQSPSSLSAFVGDKVTITCRASQSVGTYLNWYQQKAGKAPELLIYATSNLRSGVPSRFSGSGSGTDFTLTINTLQPEDFATYYCQQSYSIPRFTFGPGTKVDIK

SEQ ID NO: 223 - protein, 119 aa, Artificial Sequence
  Description of Artificial Sequence: Synthetic polypeptide
EVQLLESGGGLVQPGGSLRLSCAASGFTFSRYKMWWVRQAPGKGLEWVSGIRPSGGLTRYADSVKGRFTISRDNSKNTLYLQMNSLRAEDTAVYYCARRGDYVGGFDYWGQGTLVTVSS

SEQ ID NO: 224 - protein, 108 aa, Artificial Sequence
  Description of Artificial Sequence: Synthetic polypeptide
QDIQMTQSPGTLSLSPGERATLSCRASQSVSSSYLAWYQQKPGQAPRLLIYGASSRATGIPDRFSGSGSGTDFTLTISRLEPEDFAVYYCQHYGGSQAFGGGTKVEIK

SEQ ID NO: 225 - protein, 117 aa, Artificial Sequence
  Description of Artificial Sequence: Synthetic polypeptide
EVQLLESGGGLVQPGGSLRLSCAASGFTFSAYNMGWVRQAPGKGLEWVSSISSSGGYTGYADSVKGRFTISRDNSKNTLYLQMNSLRAEDTAVYYCARDLYRGFDYWGQGTLVTVSS

SEQ ID NO: 226 - protein, 109 aa, Artificial Sequence
  Description of Artificial Sequence: Synthetic polypeptide
QDIQMTQSPATLSVSPGERATLSCRASESVKNNLAWYQQKPGQAPRLLIYGVSTRAPGIPARFSGSGSGTDFTLTISSLEPEDFAVYYCQQRSNWPPVTFGQGTRLEIK

SEQ ID NO: 227 - protein, 124 aa, Artificial Sequence
  Description of Artificial Sequence: Synthetic polypeptide
EVQLLESGGGLVQPGGSLRLSCAASGFTFSYYGMYWVRQAPGKGLEWVSSISSSGGYTDYADSVKGRFTISRDNSKNTLYLQMNSLRAEDTAVYYCARRIKYYDIEGEGAFDIWGQGTMVTVSS

SEQ ID NO: 228 - protein, 113 aa, Artificial Sequence
  Description of Artificial Sequence: Synthetic polypeptide
QDIVMTQTPPSLPVNPGEPASISCRSSQSLLHRNGYNYLDWYLQKPGQSPQLLIHLGSYRASGVPDRFSGSGSGTDFTLKISRVEAEDVGVYYCMQPLQTPFTFGPGTKVDIK

SEQ ID NO: 229 - protein, 122 aa, Artificial Sequence
  Description of Artificial Sequence: Synthetic polypeptide
EVQLLESGGGLVQPGGSLRLSCAASGFTFSHYVMFWVRQAPGKGLEWVSRIVPSGGATMYADSVKGRFTISRDNSKNTLYLQMNSLRAEDTAVYYCARDRPLYDSSGYVDYWGQGTLVTVSS

SEQ ID NO: 230 - protein, 111 aa, Artificial Sequence
  Description of Artificial Sequence: Synthetic polypeptide
QSALTQPPSASGSPGQSVTISCTGTSSDVGAYNYVSWYQQHPDKAPKLIIYNVNERPSGVPDRFSGSKSGNTASLTVSGLQAEDEADYYCTSYAGSNKIGVSGTGTKVTVL

SEQ ID NO: 231 - protein, 128 aa, Artificial Sequence
  Description of Artificial Sequence: Synthetic polypeptide
EVQLLESGGGLVQPGGSLRLSCAASGFTFSWYPMFWVRQAPGKGLEWVSGIYSSGGPTDYADSVKGRFTISRDNSKNTLYLQMNSLRAEDTAVYYCAKDTLGRYYDFWSGYSYGMDVWGQGTTVTVSS

SEQ ID NO: 232 - protein, 105 aa, Artificial Sequence
  Description of Artificial Sequence: Synthetic polypeptide
QDIQMTQSPGTLSLSPGERATLSCRASQSVSSSYLAWYQQKPGQAPRLLIYGASSRATGIPDRFSGSGSGTDFTLTISRLEPEDFAVYYCQSGVTFGGGTKVEIK